(19) United States
(12) Patent Application Publication
(10) Pub. No.: US 2026/0174987 A1
(43) Pub. Date: Jun. 25, 2026

(54) HUMIDIFICATION CHAMBER

(71) Applicant: Fisher & Paykel Healthcare Limited, Auckland (NZ)

(72) Inventors: Michele Anne MURRAY, Auckland (NZ); Joshua LIM, Auckland (NZ); Pascal Dennis Papaioea TAANE, Auckland (NZ); Stephen David EVANS, Auckland (NZ); Leighton Yun JENSEN, Auckland (NZ); Wenjie Robin LIANG, Auckland (NZ)

(21) Appl. No.: 19/128,552

(22) PCT Filed: Nov. 15, 2023
(86) PCT No.: PCT/NZ2023/050131

§ 371 Date: May 8, 2025

Related U.S. Application Data
(60) Provisional application No. 63/383,794, filed on Nov. 15, 2022.
(60) Provisional application No. 63/476,512, filed on Dec. 21, 2022.
(60) Provisional application No. 63/517,286, filed on Aug. 2, 2023.

(51) Int. Cl. A61M 16/16; A61M 16/00; A61M 39/08

(52) U.S. Cl. CPC A61M 16/16; A61M 16/024; A61M 39/08; A61M 2205/3386; A61M 2205/3653

(57) ABSTRACT

A humidification chamber for use in respiratory or surgical humidification has a hollow body, a heat conductive body and a sealing element. The humidification chamber may be configured to be repeatedly disassembled and reassembled for reprocessing and reuse. The humidification chamber may be configured to be substituted for an alternative humidification chamber, e.g., a disposable humidification chamber, without impacting one or more functions of the humidifier such as detecting a low water or water-out condition. The heat conductive body may have one or more of a thickness, thermal conductivity, specific heat capacity, mass, or thermal capacity within a specific range.

This application claims the benefit of priority from U.S. Provisional Patent Application No. 63/383,794 , filed Nov. 15, 2022; No. 63/476,512, filed Dec. 21, 2022; and No. 63/517,286, filed Aug. 2, 2023, the entire content of each which is incorporated herein by reference.

### BACKGROUND

### Field

The present disclosure relates to a humidification chamber for respiratory or surgical humidification. More particularly, though not exclusively, the disclosure relates to a humidification chamber which is configured to be disassembled and/or reusable.

### Description of Related Art

Respiratory apparatuses are used in numerous environments including hospitals, medical facilities, residential care, palliative care, and home care to deliver pressurized respiratory gases to a patient for respiratory therapy.

Insufflation apparatuses are used in surgical environments to deliver insufflation gases to inflate or resist collapse of a body cavity, or to provide a layer of gases over exposed body parts.

The humidification of medical gases (respiratory gases or insufflation gases) may have a variety of physiological benefits for a patient.

A respiratory therapy system may include a humidification chamber to contain a volume of water which is heated by a respiratory apparatus to increase and/or maintain the humidity of a medical gas passing through the humidification chamber.

The humidification chamber may be disposable or reusable. A reusable humidification chamber should be able to be thoroughly cleaned and/or sterilized between uses. This may require repeated disassembly and reassembly of the reusable humidification chamber.

### BRIEF SUMMARY

In a first aspect there is provided a humidification chamber for respiratory or surgical humidification, comprising: a hollow body, comprising an inlet port and an outlet port; a heat conductive body, comprising a contacting wall, an upstanding wall extending from the contacting wall, and a plurality of retention protrusions extending substantially laterally from the upstanding wall; and a sealing element configured to provide a seal between the hollow body and the upstanding wall; wherein the hollow body, the heat conductive body, and the sealing element are together configured to define a chamber and the plurality of retention protrusions are configured to retain the sealing element in position about the upstanding wall.

The plurality of retention protrusions may project outwardly from the upstanding wall.

The plurality of retention protrusions may be spaced equidistantly about a perimeter of the upstanding wall.

The contacting wall may comprise a circular shape when viewed from above.

The upstanding wall may extend from a periphery of the contacting wall.

Each of the plurality of retention protrusions may extend in a radial direction.

The plurality of retention protrusions may be arranged in an upper protrusion array and a lower protrusion array.

The sealing element may be configured to be disposed between the upper protrusion array and the lower protrusion array.

The upper protrusion array may be at a first distance from a distal end of the upstanding wall, the lower protrusion array may be at a second distance from the distal end of the upstanding wall, and a difference between the first distance and the second distance may be substantially similar to a height of the sealing element.

The corresponding retention protrusions in the upper protrusion array and the lower protrusion array may be vertically aligned.

The sealing element may have a height substantially similar to the distance between the upper protrusion array and the lower protrusion array.

The plurality of retention protrusions may be arranged as an upper protrusion array and a lower singular protrusion.

The sealing element may be configured to be disposed between the upper protrusion array and the lower singular protrusion.

The upper protrusion array may be at a first distance from a distal end of the upstanding wall, the lower singular protrusion may be at a second distance from the distal end of the upstanding wall, and a difference between the first distance and the second distance may be substantially similar to a height of the sealing element.

The sealing element may have a height substantially similar to a distance between the upper protrusion array and the lower singular protrusion.

The plurality of retention protrusions may surround a perimeter of the upstanding wall.

The plurality of retention protrusions may comprise between about 2 and 96 retention protrusions, preferably between about 24 and 72 retention protrusions, and more preferably about 48 retention protrusions.

Each of the plurality of retention protrusions may be substantially semi-circular or substantially parabolic in shape.

An angle between a retention protrusion and a laterally adjacent retention protrusion may be about 15 degrees.

The angle may be configured to reduce a likelihood of the sealing element buckling during assembly and/or disassembly of the humidification chamber.

The heat conductive body may be removably attachable to the hollow body.

The heat conductive body may comprise a heat conductive body flange.

A diameter of the heat conductive body may be about 80 mm to 150 mm, and preferably about 115 mm.

A height of the upstanding wall may be about 5 mm to 20 mm, and preferably about 13 mm.

A thickness of the heat conductive body may be between about 0.2 mm to 1 mm, and preferably about 0.6 mm.

An underside of the contacting wall may be flat or convex.

A thermal capacity of the heat conductive body may be less than about 30 joules per kelvin (J/K), less than about 27 J/K, between about 16 J/K and 27 J/K, between about 19 J/K and 24 J/K, between about 20 J/K and 22 J/K, about 21 J/K, or between about 20.7 J/K and 21.2 J/K.

The heat conductive body may be anodized.

The hollow body may be substantially coaxial with the heat conductive body, when assembled.

The humidification chamber may be configured to hold up to about 500 milliliters (ml) of water, and preferably up to about 285 ml of water.

The hollow body may be substantially transparent.

The hollow body may be formed from a first polymer.

The first polymer may be a polysulfone.

The inlet port and/or the outlet port may be substantially vertical.

The hollow body may be frustoconical.

The hollow body may comprise a refill port.

The humidification chamber may comprise a refill port stopper and refill port tether.

The refill port may comprise a valve for the regulation of water supplied to the chamber through the refill port.

The humidification chamber may comprise a float assembly for the actuation of the valve.

The hollow body may comprise a hollow body flange.

The hollow body flange may project outwards from a side wall of the hollow body at or adjacent the heat conductive body, when assembled.

An upper surface of the hollow body flange and an underside of the contacting wall may be configured to engage a respiratory apparatus.

The sealing element may comprise one of an O-ring or a gasket.

The sealing element may have a toroidal shape.

The sealing element may have an inner diameter substantially similar to an outer diameter of the upstanding wall, prior to assembly.

The sealing element may have an outer diameter substantially similar to an inner diameter of the hollow body.

A height of the sealing element may be about 30% to 80% of a height of the upstanding wall, and preferably about 45% of the height of the upstanding wall.

The sealing element may be formed from a second polymer.

The second polymer may be a silicone.

The humidification chamber may be configured to withstand at least 20 reprocessing cycles, and preferably at least 50 reprocessing cycles.

A distance between a first retention protrusion and an adjacent second retention protrusion of the plurality of retention protrusions may be configured to enable a user to remove the sealing element from the heat conductive body.

The humidification chamber may comprise a removal feature to aid disassembly of the hollow body from the heat conductive body. The removal feature may comprise a slot between the heat conductive body and the hollow body. The slot may be defined by a stepped region in the hollow body. The humidification chamber may comprise an implement configured to engage the removal feature. The implement may be retained by a refill port tether.

In a second aspect there is provided a humidification chamber for respiratory or surgical humidification, comprising: a hollow body, comprising an inlet port and an outlet port; a heat conductive body; and a sealing element configured to provide a seal between the hollow body and the heat conductive body, when assembled; wherein the heat conductive body is configured to be removably attachable to the hollow body and has a thermal capacity of less than about 30 joules per kelvin (J/K), less than about 27 J/K, between about 16 J/K and 27 J/K, between about 19 J/K and 24 J/K, between about 20 J/K and 22 J/K, about 21 J/K, or between about 20.7 J/K and 21.2 J/K.

### The Heat Conductive Body May Comprise an Aluminum Alloy Material.

The heat conductive body may comprise a thickness of between about 0.2 mm and 1.2 mm, between about 0.2 mm and 1 mm, between about 0.4 mm and 0.9 mm, between about 0.6 mm and 0.8 mm, or about 0.7 mm.

The heat conductive body may comprise a thermal conductivity of between about 12 W/mK and 286 W/mK, between about 88 W/mK and 251 W/mK, between about 170 W/mK and 230 W/mK, about 227 W/mK, or between about 190 W/mK and 210 W/mK.

The heat conductive body may comprise a specific heat capacity of between about 0.5 J/gK and 1.5 J/gK, between about 0.7 J/gK and 1.1 J/gK, between about 0.8 J/gK and 1 J/gK, or about 0.9 J/gK.

The heat conductive body may comprise a mass of between about 10 g and 100 g, between about 15 g and 50 g, between about 20 g and 25 g, or about 23 g.

The humidification chamber may comprise one or more removal features configured to aid disassembly of the humidification chamber.

The humidification chamber may comprise a surround, the surround integral with, attached to, or attachable with the heat conductive body and configured to engage a base unit of a humidifier, in use.

### The Surround May Be Overmolded to the Heat Conductive Body.

The surround may comprise the one or more removal features configured to aid disassembly of the humidification chamber.

The heat conductive body may comprise a contacting wall configured to be in thermal contact with a heater plate, in use, the contacting wall comprising a uniform thickness of between about 0.2 mm and 1.2 mm, between about 0.2 mm and 1 mm, between about 0.4 mm and 0.9 mm, between about 0.6 mm and 0.8 mm, or about 0.7 mm.

The heat conductive body and/or the surround may at least in part define a channel configured to receive and retain the sealing element, in use.

In a third aspect there is provided a humidification chamber for respiratory or surgical humidification, the humidification chamber comprising: a hollow body, the hollow body comprising an inlet port and an outlet port; a heat conductive body configured to be removably attached to the hollow body, the heat conductive body comprising a contacting wall; and a surround, the surround integral with, attached to, or attachable with the heat conductive body, and comprising a removal feature; wherein the hollow body and the heat conductive body are configured to at least in part define a chamber configured to contain a volume of water, in use.

The removal feature may comprise a stepped region configured to form a slot between the surround and the hollow body when the humidification chamber is assembled, in use.

The slot may be configured for insertion of an implement.

The sealing element may be configured to be located adjacent to the heat conductive body and the surround, the sealing element configured to provide a seal between the hollow body and one or more of the heat conductive body or the surround.

The sealing element may be configured to provide a seal between the hollow body and the heat conductive body. For example, an upstanding wall of the heat conductive body.

The surround may comprise a first portion proximal to the heat conductive body and a second portion distal from the heat conductive portion, the first portion forming a shoulder configured to at least in part retain the sealing element, and the second portion comprising the removal feature.

The second portion may at least in part comprise a substantially uniform thickness of between about 2.5 mm and 3.5 mm, e.g., about 3 mm.

The second portion of the surround may be configured to act as a stop for the hollow body during assembly of the humidification chamber, in use.

A height of the removal feature may be between about 1 mm and 3 mm, e.g., about 2 mm.

A width of the removal feature may be at least about 5 mm, between about 5 mm and 100 mm, between about 5 mm and 50 mm, between about 5 mm and 30 mm, between about 10 mm and 25 mm, or between about 15 mm and 20 mm, e.g., about 16 mm. In other examples, the width of the removal feature may be between about 10 mm and 20 mm, or between about 12 mm and 16 mm, e.g., about 14 mm. In other examples, the removal feature may extend about an entire a perimeter of the humidification chamber.

The removal feature may be configured to aid disassembly of the hollow body and the heat conductive body.

The surround may comprise more than one removal feature.

The surround may comprise between one and eight, or between two and six, e.g., four, removal features.

The humidification chamber may comprise a dissimilar material to the heat conductive body.

The surround may comprise a polymer such as polypropylene.

The heat conductive body may comprise a metal or alloy such as aluminum or stainless steel.

The humidification chamber may comprise an overmold to the heat conductive body.

The humidification chamber may provide a negligible contribution to the thermal capacity of the humidification chamber.

The humidification chamber may be configured so that inserting and maneuvering (e.g., rotating and/or levering) an implement into the removal feature aids disassembly of the hollow body and the heat conductive body.

The heat conductive body and the surround, in combination, may be configured to be removably attached to the hollow body.

The heat conductive body may be configured to be removably attached to the hollow body by the surround.

The surround may be permanently connected to the heat conductive body.

The heat conductive body may comprise an upstanding wall extending from the contacting wall, wherein the surround is connected to the upstanding wall.

In a fourth aspect there is provided a breathing circuit kit comprising the humidification chamber of the first aspect, the second aspect or the third aspect, a humidifier supply tube configured to pneumatically couple with the inlet port, and an inspiratory tube configured to pneumatically couple with the outlet port.

In a fifth aspect there is provided a humidification system for respiratory or surgical humidification, comprising the humidification chamber of the first aspect, the second aspect, the third aspect, the sixth aspect or the tenth aspect; and a humidifier base unit comprising a heater plate, wherein the humidification chamber is configured to be removably received by the humidifier base unit with the contacting wall in thermal contact with the heater plate.

In a sixth aspect there is provided a heat conductive body configured to form part of a humidification chamber engageable with a humidifier base unit to deliver humidified medical gas for respiratory or surgical treatment, wherein the heat conductive body is configured to be removably attached to a polymer body to form the humidification chamber, and the heat conductive body has a thermal capacity of less than 30 joules per kelvin (J/K), less than about 27 J/K, between about 16 J/K and 27 J/K, between about 19 J/K and 24 J/K, between about 20 J/K and 22 J/K, about 21 J/K, or between about 20.7 J/K and 21.2 J/K.

In a seventh aspect there is provided a heat conductive body configured to form part of a humidification chamber engageable with a humidifier base unit to deliver humidified medical gas for respiratory or surgical treatment, the heat conductive body comprising a contacting wall configured to thermally contact a heater plate of a humidifier base unit, an upstanding wall extending from the contacting wall, and a plurality of retention protrusions extending substantially laterally from the upstanding wall.

In an eighth aspect there is provided a humidification system for respiratory or surgical humidification, comprising: a humidifier base unit comprising a heater plate; a first humidification chamber configured to be engageable with the humidifier base unit in thermal contact with the heater plate; and a second humidification chamber configured to be engageable with the humidifier base unit in thermal contact with the heater plate; wherein the first humidification chamber and the second humidification chamber differ structurally, and a thermal capacity of the second humidification chamber is substantially equivalent to a thermal capacity of the first humidification chamber.

The first humidification chamber may comprise a disposable humidification chamber and the second humidification chamber may comprise a reusable humidification chamber. The reusable humidification chamber may be configured to be disassembled and reassembled, and the disposable humidification chamber may be permanently assembled.

The thermal capacity of the second humidification chamber may be within 15%, within 10%, within 5%, or within 2.5% of the thermal capacity of the first humidification chamber.

The thermal capacity of the second humidification chamber may be less than 30 joules per kelvin (J/K), less than about 27 J/K, between about 16 J/K and 27 J/K, between about 19 J/K and 24 J/K, between about 20 J/K and 22 J/K, about 21 J/K, or between about 20.7 J/K and 21.2 J/K.

The second humidification chamber may comprise the humidification chamber of the first aspect, the second aspect, the third aspect, the sixth aspect, or the tenth aspect.

In a ninth aspect there is provided a humidification system for respiratory or surgical humidification, comprising: a humidifier base unit comprising a heater plate; a first humidification chamber configured to be engageable with the humidifier base unit in thermal contact with the heater plate; and a second humidification chamber configured to be engageable with the humidifier base unit in thermal contact with the heater plate; wherein the first humidification chamber and the second humidification chamber differ structurally, and the second humidification chamber is configured to be substitutable for the first humidification chamber without adjusting a parameter or algorithm of the humidifier base unit.

The first humidification chamber and the second humidification chamber may be configured to provide a similar level of humidity to a medical gas with the same input conditions.

The first humidification chamber and the second humidification chamber may be configured to provide a similar level of humidity to a medical gas at an equivalent steady state heater plate power, in use.

The humidifier base unit may be configured to detect a low water or water-out condition.

The humidifier base unit may be configured to determine a response of the first humidification chamber or the second humidification chamber, plus a volume of water (if any) contained therein, to a characteristic energization signal applied to the heater plate.

A thermal capacity of the second humidification chamber may be substantially equivalent to a thermal capacity of the first humidification chamber.

The second humidification chamber may comprise the humidification chamber of the first aspect, the second aspect or the third aspect.

In a tenth aspect there is provided a humidification chamber for use with a humidifier base unit to humidify a flow of medical gases for respiratory therapy or surgical insufflation, the humidification chamber comprising: a hollow body comprising an inlet port for ingress of the flow of medical gases into the humidification chamber, in use, and an outlet port for egress of the flow of medical gases from the humidification chamber, in use; a heat conductive body, the hollow body and the heat conductive body in combination configured to form, at least in part, a chamber configured to receive a volume of liquid with a headspace providing a passage for the flow of medical gases from the inlet port to the outlet port; and a sealing element configured to provide a seal between the hollow body and the heat conductive body.

The heat conductive body may comprise an aluminum alloy material.

The heat conductive body may comprise a thickness of between about 0.2 mm and 1.2 mm, between about 0.2 mm and 1 mm, between about 0.4 mm and 0.9 mm, between about 0.6 mm and 0.8 mm, or about 0.7 mm.

The heat conductive body may comprise a thermal conductivity of between about 12 W/mK and 286 W/mK, between about 88 W/mK and 251 W/mK, between about 170 W/mK and 230 W/mK, about 227 W/mK, or between about 190 W/mK and 210 W/mK.

The heat conductive body may comprise a specific heat capacity of between about 0.5 J/gK and 1.5 J/gK, between about 0.7 J/gK and 1.1 J/gK, between about 0.8 J/gK and 1 J/gK, or about 0.9 J/gK.

The heat conductive body may comprise a mass of between about 10 g and 100 g, between about 15 g and 50 g, between about 20 g and 25 g, or about 23 g.

The heat conductive body may comprise a thermal capacity of less than about 30 J/K, less than about 27 J/K, between about 16 J/K and 27 J/K, between about 19 J/K and 24 J/K, between about 20 J/K and 22 J/K, about 21 J/K, or between about 20.7 J/K and 21.2 J/K.

At least the hollow body and the heat conductive body may be configured to be repeatedly assembled with, and disassembled from, each other.

The humidification chamber may comprise one or more removal features configured to aid disassembly of the humidification chamber.

The humidification chamber may comprise a surround, the surround integral with, attached to, or attachable with the heat conductive body and configured to engage a base unit of the humidifier, in use.

The surround may comprise one or more removal features configured to aid disassembly of the humidification chamber.

The heat conductive body may comprise a contacting wall configured to be in thermal contact with a heater plate of a base unit of the humidifier when the humidification chamber is engaged with the base unit, in use, the contacting wall comprising the thickness of between about 0.2 mm and 1.2 mm, between about 0.2 mm and 1 mm, between about 0.4 mm and 0.9 mm, between about 0.6 mm and 0.8 mm, or about 0.7 mm.

The heat conductive body may at least in part define a channel configured to receive and retain the sealing element, in use.

In an eleventh aspect, there is provided a humidification circuit kit comprising the humidification chamber of the first aspect, the second aspect, the third aspect, the sixth aspect or the tenth aspect, a humidifier supply tube configured to be coupled with the inlet port, and an inspiratory tube or an insufflation delivery tube configured to be coupled with the outlet port.

In a twelfth aspect, there is provided a humidifier system configured to humidify a flow of medical gases for respiratory therapy or surgical insufflation, the humidifier system comprising: the humidification chamber of the tenth aspect; an alternative humidification chamber, the alternative humidification chamber differing structurally from the humidification chamber; and a base unit comprising a heater plate and a controller, the base unit configured to removably receive either of the humidification chamber and the alternative humidification chamber in thermal contact with the heater plate, the heater plate configured to heat the humidification chamber or the alternative humidification chamber to heat and humidify the flow of medical gases passing through the humidification chamber or the alternative humidification chamber, in use, and the controller configured to control the heater plate and to detect one or more of a low water condition or a water-out condition without identification (e.g., manual identification or automatic identification) of the humidification chamber or the alternative humidification chamber.

The humidification chamber may comprise a reusable humidification chamber and the alternative humidification chamber may comprise a disposable humidification chamber.

In a thirteenth aspect, there is provided a humidifier system configured to humidify a flow of medical gases for respiratory therapy or surgical insufflation, the humidifier system comprising: a base unit comprising a heater plate configured to generate heat, in use; a disposable humidification chamber configured to be removably received by the base unit in thermal contact with the heater plate; and a reusable humidification chamber configured to be removably received by the base unit in thermal contact with the heater plate, wherein each of the disposable humidification chamber and the reusable humidification chamber comprise a heat conductive body with a thermal capacity between about 16 J/K and 27 J/K, between about 19 J/K and 24 J/K, between about 20 J/K and 22 J/K, or about 21 J/K.

The base unit may comprise a controller configured to detect one or more of a low water condition or a water-out condition based on a response of the disposable humidification chamber or the reusable humidification chamber, including any liquid contained therein, to a characteristic energization signal applied to the heater plate.

Further aspects, novel features and/or advantages will be readily apparent to those skilled in the art in light any one or more of the illustrative examples set out in the following detailed description and drawings.

### BRIEF DESCRIPTION OF THE DRAWINGS

FIG. 1 illustrates an example of a respiratory apparatus.

FIG. 2 illustrates a schematic diagram of a respiratory therapy system in use.

FIG. 3 is a perspective view of a humidification chamber in accordance with a first example.

FIG. 4 is an exploded view of the humidification chamber in accordance with the first example.

FIG. 5 is a side view of the humidification chamber in accordance with the first example.

FIG. 6 is a cross-section of the humidification chamber in accordance with the first example.

FIG. 7 is a perspective view of a heat conductive body of the humidification chamber in accordance with the first example.

FIG. 8 is a top view of the heat conductive body in accordance with the first example.

FIG. 9 is a side view of the heat conductive body in accordance with the first example.

FIG. 10 is a detailed view of the heat conductive body in accordance with the first example.

FIG. 11 is a cross-section of the heat conductive body in accordance with the first example.

FIG. 12 is a cutaway illustration of the displacement of a sealing element during attachment of a hollow body to the heat conductive body of the humidification chamber in accordance with the first example.

FIG. 13 is a perspective view of a heat conductive body in accordance with a second example.

FIG. 14 is a top view of the heat conductive body in accordance with the second example.

FIG. 15 is a side view of the heat conductive body in accordance with the second example.

FIG. 16 is a perspective view of a heat conductive body in accordance with a third example.

FIG. 17 is a top view of the heat conductive body in accordance with the third example.

FIG. 18 is a side view of the heat conductive body in accordance with the third example.

FIG. 19 is a perspective view of a heat conductive body in accordance with a fourth example.

FIG. 20 is a top view of the heat conductive body in accordance with the fourth example.

FIG. 21 is a side view of the heat conductive body in accordance with the fourth example.

FIG. 22 is a perspective view of a heat conductive body in accordance with a fifth example.

FIG. 23 is a top view of the heat conductive body in accordance with the fifth example.

FIG. 24 is a side view of the heat conductive body in accordance with the fifth example.

FIG. 25 is a perspective view of a heat conductive body and a surround in accordance with a sixth example.

FIG. 26 is a top view of the heat conductive body and the surround in accordance with the sixth example.

FIG. 27 is a cross-section view of the heat conductive body and the surround in accordance with the sixth example.

FIG. 28 is a cross-section view of the heat conductive body and the surround in accordance with the sixth example.

FIG. 29 is a front view of the heat conductive body and the surround in accordance with the sixth example.

FIG. 30 is a perspective view of the heat conductive body and the surround in accordance with the sixth example.

FIG. 31 is a detailed view of the heat conductive body and the surround in accordance with the sixth example.

FIG. 32 is a bottom view of the heat conductive body and the surround in accordance with the sixth example.

FIG. 33 is a perspective view of a humidifier comprising a base unit and a humidification chamber in accordance with a second example, during a mounting process.

FIG. 34 is a perspective view of the humidifier of FIG. 33, with the humidification chamber mounted on the base unit.

FIG. 35 is a Front View of the Humidifier of FIG. 34.

FIG. 36 is a front view of a humidification chamber in accordance with a third example.

FIG. 37 is a detailed cross-section of the humidification chamber in accordance with the third example.

### DETAILED DESCRIPTION

### System Overview

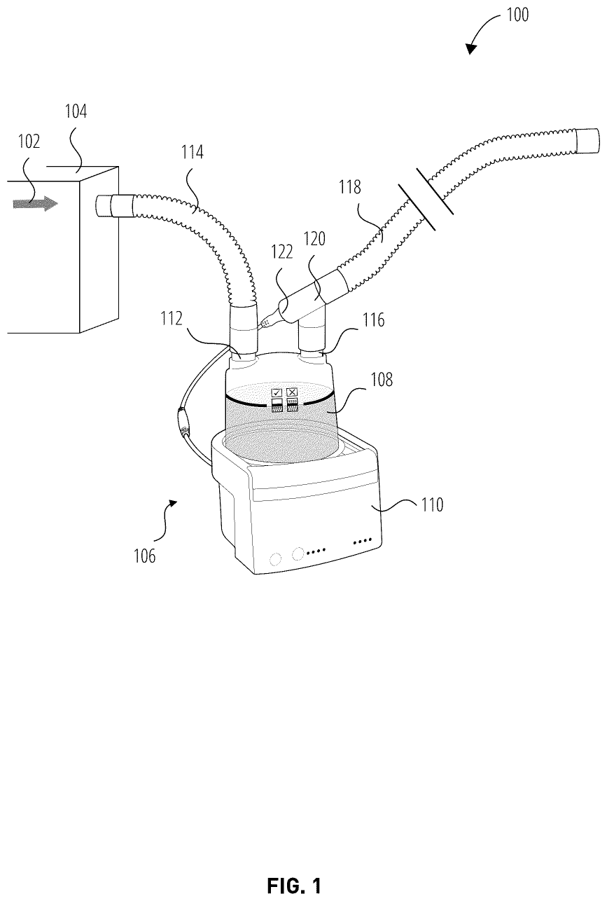

With reference to FIG. 1, provided is an example of a respiratory apparatus 100. A gases source 102 supplies a medical gas. The medical gas may include, for example, air, medical air, oxygen, carbon dioxide, nitrogen, or mixtures thereof. The medical gas is supplied to a flow generator 104. The flow generator 104 supplies the medical gas to a humidifier 106. The humidifier 106 heats and humidifies the medical gas for use in respiratory therapy. In some examples, the gases source 102 may be a wall source, a compressed gas cylinder, or ambient air. The flow generator 104 may control one or more of the pressure, volume, or flow rate of the medical gas supplied to the humidifier 106. In other examples, the flow generator 104 and the humidifier 106 may be integrated in a single device (i.e., within a single housing).

The humidifier 106 may comprise a humidification chamber 108 and a base unit 110. The base unit 110 may comprise a heater plate, control electronics, and a user interface. The base unit 110 is configured to receive a humidification chamber 108 that holds a volume of water. In use, the medical gas is passed over a headspace within the humidification chamber 108 above the volume of water. The humidification chamber 108 may be removable from a base unit 110 of the humidifier 106.

The humidification chamber 108 may comprise a hollow body and a heat conductive body. The hollow body may comprise a top wall and one or more side walls, with an open end opposing the top wall. In the illustrated examples, the heat conductive body provides a base for the humidification chamber 108. In some examples, the heat conductive body may be a side wall of the humidification chamber.

The heat supplied by the humidifier 106, e.g., the base unit 110, to the heat conductive body controls, e.g., increases, the temperature of the volume of water within the humidification chamber 108, in use. And also controls, e.g., increases, the temperature and humidity of the medical gas that passes through the headspace of the humidification chamber 108.

The humidification chamber 108 comprises an inlet port 112 that connects to a humidifier supply tube 114 and receives the medical gas from the flow generator 104. The humidification chamber 108 comprises an outlet port 116 which connects to an inspiratory tube 118 for delivery of the medical gas to a patient via a patient interface (not shown). The inspiratory tube 118 may comprise a tube connector 120 for pneumatic coupling with the outlet port 116. The inspiratory tube 118 may be heated to increase or maintain the heat and/or humidity of the medical gas as it is conveyed from the humidification chamber 108 towards the patient. The inspiratory tube 118 may be heated by a heater within the lumen of the tube, embedded within the tube wall, or surrounding the tube along at least a portion of its length. In some examples, the heater may comprise a resistive heater wire, e.g., located within the lumen or embedded in the tube wall. The inspiratory tube 118 may comprise an electrical connector 122 to receive power for the heater wire from the flow generator 104 or the humidifier 106. In some examples, the electrical connector may comprise a socket or plug. In some examples, the electrical connector may be integrated as part of a tube connector 120. In other examples, the inspiratory tube 118 may comprise a water jacket and be heated by circulation of heated water.

In some examples, the inspiratory tube may instead be a delivery tube for supplying humidified medical gas for surgical procedures.

The humidifier supply tube 114, humidification chamber 108, and inspiratory tube 118 may each be considered respiratory apparatus components together forming a breathing circuit. The respiratory apparatus components of a breathing circuit, including the humidification chamber 108, may be sold individually or packaged together as a breathing circuit kit.

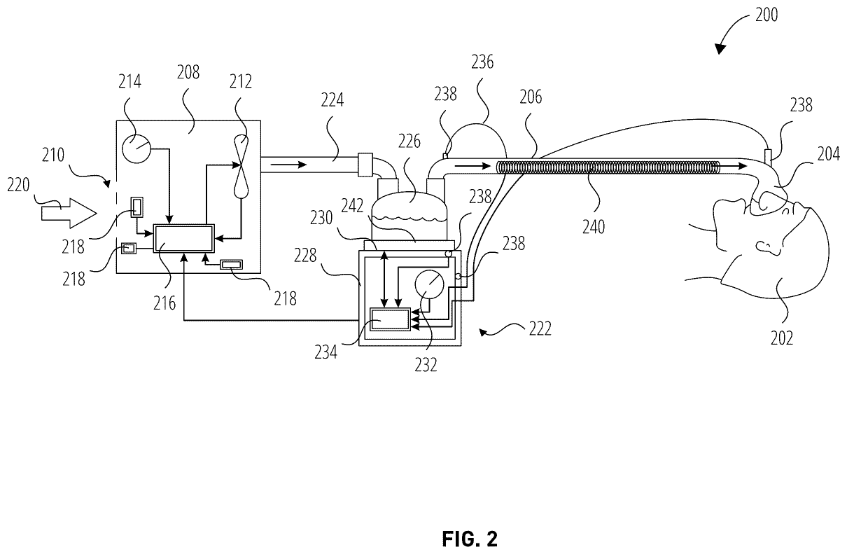

With reference to FIG. 2, provided is a schematic diagram of a respiratory therapy system 200 providing respiratory therapy to a patient 202. The illustrated respiratory therapy system 200 provides an example of a single-limb breathing circuit. Gases expired by the patient 202, and any excess medical gas supplied to the patient 202, may be vented to atmosphere.

In other examples, a respiratory therapy system may comprise a dual-limb breathing circuit. The dual-limb breathing circuit further includes an expiratory tube to convey the expired gas and excess medical gas away from the patient interface 204. The expiratory tube may be configured to convey the expired gas and excess medical gas to a return inlet of the flow generator. The expiratory tube may be heated, e.g., to maintain the heat and/or humidity of the expired gas and any excess medical gas not inspired by the patient. The dual-limb breathing circuit may further include a wye-piece. The wye-piece may be configured to fluidly couple the inspiratory tube 206, the patient interface 204 and the expiratory tube.

The breathing circuit and/or breathing circuit kit may also include further respiratory apparatus components such as one or more of an expiratory tube, a water trap, a wye-piece, and/or any number of connectors or adaptors (not shown).

The flow generator illustrated in FIG. 2 is a ventilator 208. The ventilator 208 may be configured to control the pressure, volume, or flow rate of medical gas delivered to a patient 202. The patient 202 is connected to a patient interface 204. The respiratory therapy may be non-invasive ventilation (NIV) or continuous positive airway pressure (CPAP) therapy. The patient interface 204 may comprise a non-invasive sealing interface such as a total-face mask, full-face mask, nasal mask, or pillows interface. In some examples, the respiratory therapy may be invasive ventilation. The patient interface may comprise an invasive interface such as an endotracheal tube or tracheostomy tube. In some examples, the respiratory therapy may be nasal high flow (NHF) therapy. The patient interface may comprise a non-sealing interface such as a non-sealing nasal cannula.

The ventilator 208 comprises a gases inlet 210, a flow source 212, a ventilator user interface 214, a ventilator controller 216, and one or more ventilator sensors 218. The gases inlet 208 is configured for connection to a gases source 220. The gases source 220 is represented by an arrow in FIG. 2, but in some examples may include one or more of a wall source, a compressed gas cylinder, or ambient air. The one or more ventilator sensors 218 may be used to detect characteristics of the medical gas. The ventilator controller 216 may control the flow source 212 to provide a set flow rate, pressure, or volume of medical gas dependent on settings, e.g., set using the ventilator user interface 214.

The ventilator 208 supplies the humidifier 222 with the medical gas via the humidifier supply tube 224.

The humidifier 222 comprises a humidification chamber 226 and a base unit 228. The humidifier 222, e.g., base unit 228, comprises a heater plate 230, a humidifier user interface 232, a humidifier controller 234, an electrical lead 236, and one or more humidifier sensors 238. The humidifier sensors 238 may include one or more of a heater plate temperature sensor, an ambient air temperature sensor, a chamber outlet temperature sensor, and a patient end temperature sensor. The electrical lead 236 may connect the resistive heater wire 240 within the inspiratory tube 206 to the humidifier 222 for the supply of power under control of the humidifier controller 234. The electrical lead 236 may further connect the chamber outlet temperature sensor to the humidifier controller 234. A further electrical lead may connect the patient end temperature sensor with the humidifier controller 234.

The heater plate 230 supplies heat to the heat conductive body 242 of the humidification chamber 226 under control of the humidifier controller 234. The one or more humidifier sensors 238 may be used to detect characteristics of one or more of the medical gas, the humidification chamber 226, or the base unit 228, e.g., heater plate 230. The humidifier controller 234 may control the temperature and/or humidity of the medical gas dependent on the settings, e.g., set using the humidifier user interface 232 (e.g., a set temperature and/or a set humidity). The temperature of the medical gas may be between 26° Celsius (C) and 43° C., and preferably between about 29° C. and 40° C., e.g., at the point of delivery to the patient. The absolute humidity of the medical gas may be greater than about 12 mg/L, and preferably greater than about 33 mg/L. The relative humidity of the medical gas may be greater than about 90%, preferably greater than about 95%, and more preferably about 100%. The temperature and/or humidity of the medical gas may be dependent on the respiratory therapy.

The humidification chamber 226 may be removably received by the base unit 228 in a horizontal direction. In such a configuration, the humidification chamber 226 may be described as a slide-on humidification chamber. In other examples, the humidification chamber may be received by the humidifier in a vertical direction. In such a configuration, the humidification chamber may be described as a drop-in humidification chamber. In some examples, the inlet port and/or the outlet port of the slide-on or drop-in humidification chamber may be configured to establish pneumatic connections with the base unit 228 as the humidification chamber is received by the base unit. In some examples, the base unit 228 may comprise a compartment configured to receive and at least partially enclose the humidification chamber. In some examples, the compartment may be closed and/or substantially sealed by a lid or door of the humidifier.

The heater plate 230 and the humidification chamber 226 may be biased towards each other to maintain thermal contact. In some examples, the heater plate 230 may be sprung or otherwise resiliently biased to provide an upwards biasing force against the bottom of the humidification chamber 226. The humidification chamber 226 may be braced against this upwards biasing force. In other examples, the humidifier and/or humidification chamber may be configured to provide a downwards biasing force against a fixed heater plate, or both the heater plate and the humidification chamber may be biased towards each other.

### Water-Out Detection

The humidification chamber 226 may be filled with a volume of water by a user ahead of the initiation of respiratory therapy. In some examples, a humidification chamber may be supplied with water throughout respiratory therapy to reach or maintain a set water level. The humidification chamber 226 may be adapted to hold a maximum volume of water dependent on the patient population for which the humidification chamber is adapted. The maximum volume of water that a humidification chamber 226 can hold may be an important characteristic for the performance of the respiratory therapy system 200. During the provision of respiratory therapy, the volume of water within the humidification chamber 226 may decrease such that the humidification chamber is in a low water or water-out condition. The low water or water-out condition may impact performance of the respiratory therapy system 200 and reduce the humidity of the medical gas to below the set humidity.

The humidifier 222 may be configured to detect one or more properties of the humidification chamber 226 or its contents. In one example, the humidifier 222 may detect when the humidification chamber 226 has reached a low water or water-out condition. An example system and method for determining a low water or water-out condition for a humidification chamber is described in United States Patent Application Publication No. 2022/0008670, the entirety of which is incorporated herein by reference. An example system and method for determining a low water or water-out condition for a humidification chamber 226 may comprise inferring the specific heat capacity of the humidification chamber (including the volume of water contained therein). Inferring the specific heat capacity of the humidification chamber 226 may comprise applying a characteristic energization signal to the heater plate 230 and detecting a response of the humidification chamber 226 (including any water or other humidification liquid contained therein). The characteristic energization signal may comprise a supplementary signal applied to a heater plate power signal. The characteristic energization signal may be at a predetermined frequency that is different (such as being higher or lower) than a conventional heater plate power signal. The response may comprise one or more of a magnitude or a phase of a signal indicative of a response to the characteristic energization signal. The signal indicative of a response to the characteristic energization signal may comprise a temperature signal from a temperature sensor at or near the heater plate 230. The response may be indicative of a specific heat capacity, the value of which will vary dependent on the volume of water contained by the humidification chamber 226. The specific heat capacity of the humidification chamber 226 may decrease as water is removed from the humidification chamber 226. A low water or water-out condition may be determined when the response crosses a threshold value. The determination of the low water or water-out condition may be impacted by the respiratory therapy, the flow rate of the medical gas, the set temperature, and/or the set humidity. Properties of the humidification chamber 226 may also impact the determination of the low water or water-out condition. The mass, thermal capacity and/or thermal conductivity of the humidification chamber may impact the determination of the low water or water-out condition. Determination of the low water or water-out condition may be hindered by a humidification chamber with a large mass, large thermal capacity and/or low thermal conductivity. For example, a humidification chamber 226 with a relatively large mass, in comparison to the maximum volume of water, may attenuate the energization signal and/or impact the detection of the response.

### Reusability Considerations

To reduce the transmission of pathogens between patients 202, and for reasons of cleanliness, respiratory apparatus components should be either reprocessed or replaced between subsequent uses of the respiratory therapy system 200. Respiratory apparatus components may be adapted for reuse (i.e., reusable) or disposal (i.e., consumable). To enable the reuse of respiratory apparatus components, appropriate standards of cleanliness should be attainable following a reprocessing cycle. A reprocessing cycle may comprise cleaning (e.g., in a home care environment) or high-level disinfection (e.g., in a hospital environment). High-level disinfection may comprise use of a washer disinfector or an autoclave. The effectiveness of a reprocessing cycle may be dependent on cleaning fluids contacting surfaces which may have been exposed to any potential pathogens/contaminants during the provision of respiratory therapy. Pathogens/contaminants may comprise any one or more of bacteria, viruses, germs, dirt, or bodily fluids. To improve the effectiveness of a reprocessing cycle, respiratory apparatus components may be designed to reduce areas where pathogens/contaminants may accumulate.

A humidification chamber which can be disassembled (e.g., a hollow body removably attachable to the heat conductive body) may be more likely to attain an appropriate standard of cleanliness following a reprocessing cycle. A humidification chamber which can be disassembled may enable greater access to interior surfaces of the humidification chamber which may have been exposed to pathogens/contaminants during the provision of respiratory therapy.

A humidification chamber which can be disassembled may comprise a heat conductive body which can be disassembled from one or more other parts of the humidification chamber. For example, the heat conductive body may be detached from the hollow body.

The reuse of respiratory apparatus components may be beneficial from a perspective of reducing waste and greenhouse gas emissions. The reuse of respiratory apparatus components may reduce the emissions associated with one or more of the manufacture, shipping and handling, and/or end-of-life (e.g., disposal) of respiratory apparatus components.

A humidification chamber which can be disassembled may be beneficial for reducing end-of-life emissions by enabling separation of the parts of the humidification chamber to be recycled.

A humidification chamber adapted to be reusable may have an associated increased weight to withstand a repeated use and reprocessing cycles (i.e., when compared to a disposable humidification chamber, which may be designed for a shorter lifespan). Reducing the weight of a humidification chamber may reduce the emissions associated with the shipping and handling of respiratory apparatus components. Reducing the weight of a humidification chamber may improve the usability or performance thereof. Reducing the weight of a humidification chamber may reduce the time required at the initiation of respiratory therapy for the medical gas to reach the set temperature and/or set humidity. Reducing the weight of a humidification chamber may improve the responsiveness of the humidification chamber for respiratory therapy (i.e., the humidification chamber may more quickly reach the set temperature and/or set humidity). Reducing the weight of a humidification chamber may improve consistency of performance of the humidifier when used with both reusable humidification chambers and disposable humidification chambers. Reducing the weight of a humidification chamber may improve reliability of one or more functions of the humidifier, e.g., a low water or water-out detection function. Improving the ease by which a chamber is disassembled may be beneficial for a user.

### First Example Humidification Chamber

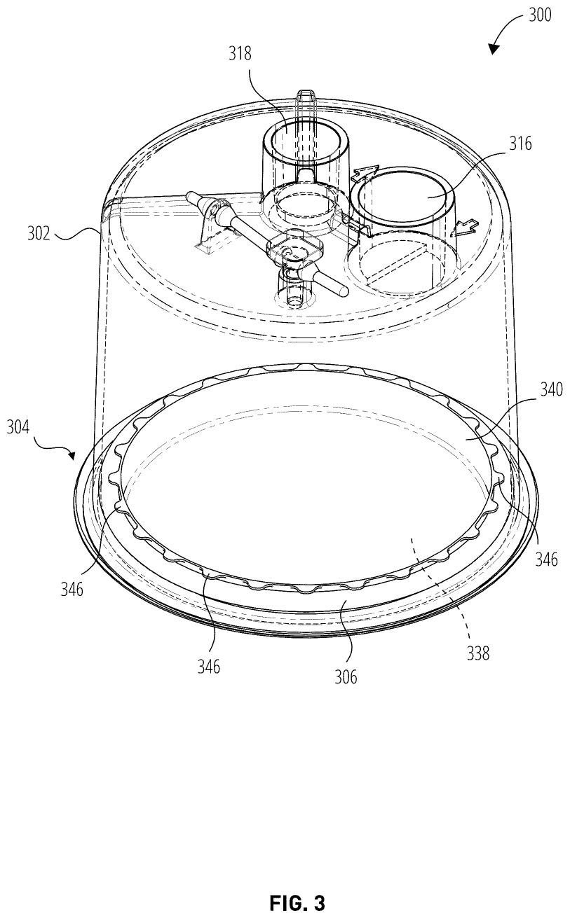

With reference to FIG. 3, provided is a first example of a humidification chamber 300 for use in respiratory therapy. The humidification chamber 300 broadly comprises a hollow body 302, a heat conductive body 304 and a sealing element 306, each described in further detail below. The hollow body 302, the heat conductive body 304, and the sealing element 306 are together configured to define a chamber when assembled as shown in FIG. 3.

The humidification chamber 300 may be used in place of the humidification chamber 108 in the respiratory apparatus 100 of FIG. 1, or the humidification chamber 226 in the respiratory therapy system 200 of FIG. 2.

### Hollow Body

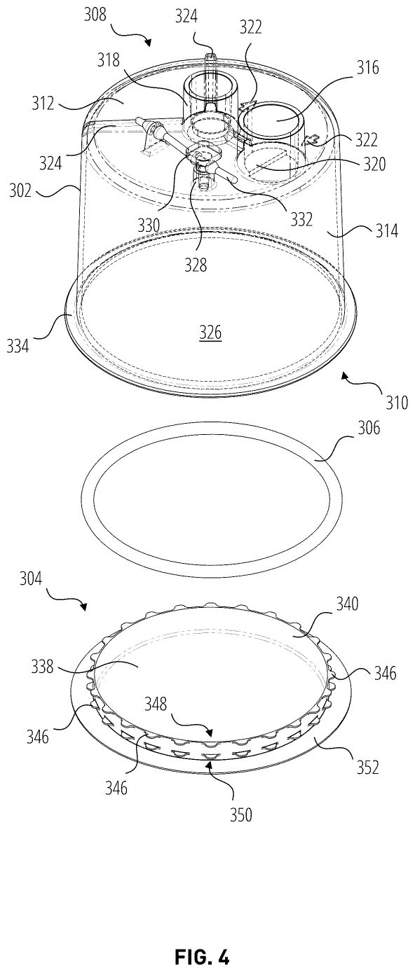

With reference to FIG. 4, provided is an exploded view of the humidification chamber 300.

The hollow body 302 has a first end 308 and a second end 310. The hollow body 302 may comprise a top wall 312 at the first end 308 and one or more side walls 314 extending from the top wall 312 towards the open second end 310.

The first end 308 may comprise an inlet port 316 and/or an outlet port 318. The inlet port 316 and the outlet port 318 may be dissimilar, e.g., different inner and/or outer diameters. In some examples, the inlet port 316 and/or the outlet port 318 may be located on the one or more side walls 314. In some examples, the inlet port 316 and/or the outlet port 318 may be defined, at least in part, by the heat conductive body 304.

The inlet port 316 is configured to connect to a humidifier supply tube 114, 224. The outlet port 318 is configured to connect to an inspiratory tube 118, 206. The inlet port 316 and/or outlet port 318 may be substantially parallel and/or substantially vertical, as shown. In some examples, as shown in FIG. 3, the inlet port 316 may be offset from a center of the top wall 312. In some examples, also as shown in FIG. 3, the outlet port 318 may be disposed at about a center of the top wall 312. In other examples, both the inlet port 316 and the outlet port 318 may be offset from the center of the top wall 312, e.g., disposed at, or towards, opposing sides of the top wall 312. The inlet port 316 and/or outlet port 318 may be substantially cylindrical or frustoconical. The inlet port 316 and/or outlet port 318 may comprise a male taper and/or a female taper. The inlet port 316 and/or the outlet port 318 may have a standard medical taper suitable for connection to a variety of humidifier supply tubes or inspiratory tubes, respectively. The inlet port 316 may comprise a complementary taper of the outlet port 318 thereby allowing the humidifier supply tube 114, 224 to connect directly to the inspiratory tube 118, 206 during use, bypassing the humidification chamber 300, to ameliorate interruption of respiratory therapy during inspection, replacement or filling of the humidification chamber 300. The inlet port 316 may comprise a baffle 320. The baffle 320 may influence the direction of the flow of medical gas at the inlet port 316. In some examples, the baffle 320 may be configured to ameliorate the flow of gases directly from the inlet port 316 to the outlet port 318, and instead increase a dwell time within the headspace of the humidification chamber 300 to increase uptake of water vapor by the medical gas. The baffle 320 may partially occlude the inlet port 316. The baffle 320 may be provided in a plane which is substantially perpendicular to an axis of the inlet port 316, as illustrated. In other examples, the baffle 320 may be provided in a plane which is inclined with respect to the axis of the inlet port 316.

The hollow body 302 may comprise one or more indicia 322. The one or more indicia 322 may indicate the use of the inlet port 316 and/or outlet port 318. The one or more indicia 322 may also include a fill line (not shown) on the side wall 314 indicating a maximum water level. The fill line may represent a proportion of the overall volume of the humidification chamber 300 to allow for a headspace and circulation of the medical gas.

The hollow body 302 may comprise one or more chamber ribs 324. The chamber ribs 324 may be configured to strengthen and/or stiffen the hollow body 302. In some examples, a chamber rib 324 may project outwardly from a portion of the outer surface of the top wall 312 and/or the side wall 314. In some examples, a chamber rib 324 may project inwardly from a portion of the inner surface of the top wall 312 and/or the side wall 314. A chamber rib 324 (or ribs) provided on the top wall 312 may extend in a substantially radial direction from a central outlet port 318 to, or towards, the side wall 314. In other examples, one or more chamber ribs 324 on the side wall 314 may extend in a substantially longitudinal direction, from (or near) the first end 308 towards the second end 310.

The second end 310 may comprise an opening 326 for receipt of the heat conductive body 304. The hollow body 302 at the second end 310 may comprise a shape complementary to the heat conductive body 304.

The hollow body 302 may be frustoconical or cylindrical. The hollow body 302 may be formed from a first polymer. The first polymer may be chosen to withstand a reprocessing cycle without distortion and/or degradation due to heat and/or chemicals. The first polymer may comprise polysulfone. The hollow body 302 may be substantially transparent. The hollow body 302 being substantially transparent may enable a user to observe the volume of water within the humidification chamber 300 and reduce the likelihood of an unexpected low water or water-out condition.

The hollow body 302 may further comprise a refill port 328. The refill port 328 may be used to increase or maintain the volume of water within the humidification chamber 300 in use. The humidification chamber 300 may further comprise a refill port stopper 330, and a refill port tether 332. The refill port stopper 330 may be used to selectively occlude the refill port 328.

The humidification chamber 300 may be configured to hold up to about 500 milliliters (ml) of water, such as up to about 285 ml of water, while maintaining a headspace for circulation of the medical gas. In some examples, the refill port 328 may comprise a valve (not shown) for the regulation of water supplied to the humidification chamber 300 through the refill port 328. In some examples, the humidification chamber 300 may comprise a float assembly (not shown) for actuation of the valve. In some examples, the humidification chamber 300 and/or breathing circuit kit may further comprise a water tube and a spike configured to fluidly couple the refill port 328 with a water source, e.g., a sterile water bag. Water may be supplied to the refill port, in use, by gravity or a pump.

The hollow body 302 may comprise a hollow body flange 334 at the second end 310. The hollow body flange 334 may extend radially outwardly from the side wall 314. The hollow body flange 334 may form an annulus about the side wall 314. The hollow body flange 334 may be provided in a plane which is parallel to the top wall 312. The hollow body flange 334 may be configured to strengthen and/or stiffen the hollow body 302 at the second end 310. The hollow body flange 334 may be configured to act as a stop for the heat conductive body 304 (or vice versa) during assembly of the humidification chamber 300. The hollow body flange 334 may be configured to engage a base unit of a humidifier, e.g., to retain the humidification chamber 300 and/or to brace the humidification chamber 300 against an upwards biasing force.

With continued reference to FIG. 4, the humidification chamber 300 is separable and can be disassembled into a disassembled configuration. The sealing element 306 may be removably attached to the heat conductive body 304. The heat conductive body 304 may be removably attached to the hollow body 302. In some examples, however, the heat conductive body 304 and/or the sealing element 306 may be integral with the hollow body 302. Or the sealing element 306 may be integral with the heat conductive body 304.

The center of the hollow body 302 may be substantially coaxial with the center of the heat conductive body 304, during or after assembly.

In some examples, one or more of the hollow body 302, the heat conductive body 304, and the sealing element 306 are configured to be reusable. In some examples, each of the hollow body 302, the heat conductive body 304, and the sealing element 306 are configured to be reusable.

The disassembled configuration of the humidification chamber 300 may improve the likelihood the humidification chamber 300 can attain an appropriate standard of cleanliness following a reprocessing cycle.

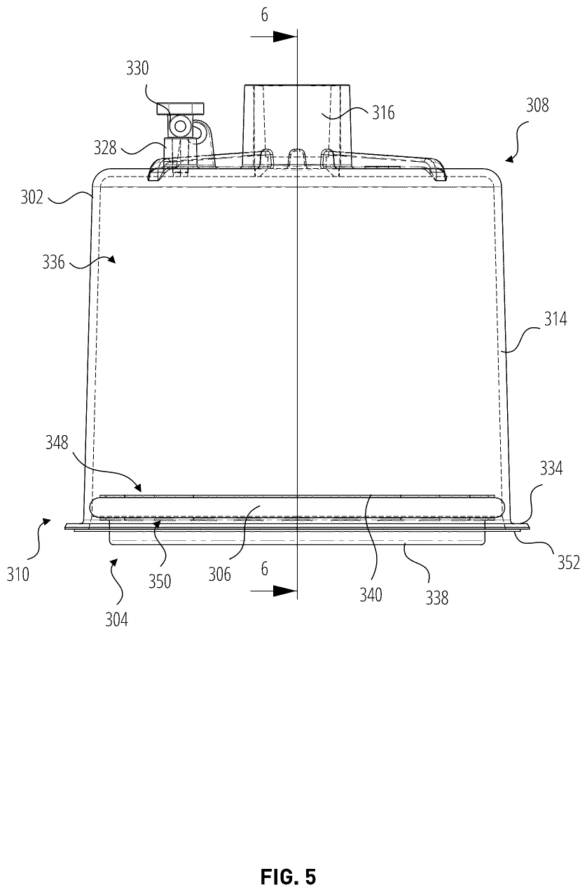

As shown FIG. 5, the hollow body 302 and the heat conductive body 304 together define a chamber 336 for receiving a volume of water and a flow of medical gas.

The side wall 314 may be tapered slightly from the second end 310 towards the first end 308. That is, the side wall 314 may be wider at, or towards, the second end 310 than it is at, or towards, the first end 308. This may give the hollow body 302 a slightly frustoconical shape.

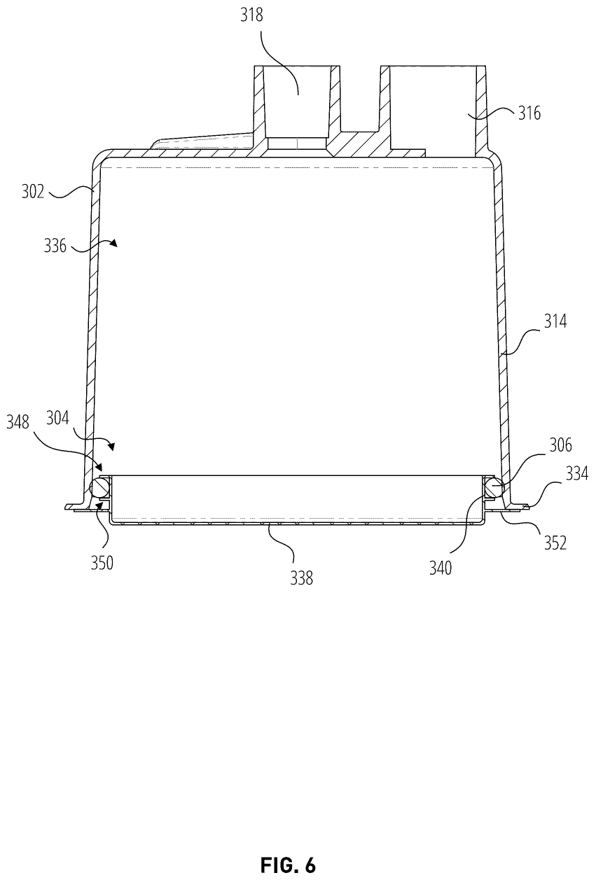

FIG. 6 illustrates a cross-section of the humidification chamber 300 through the plane 6-6 shown in FIG. 5.

### Heat Conductive Body

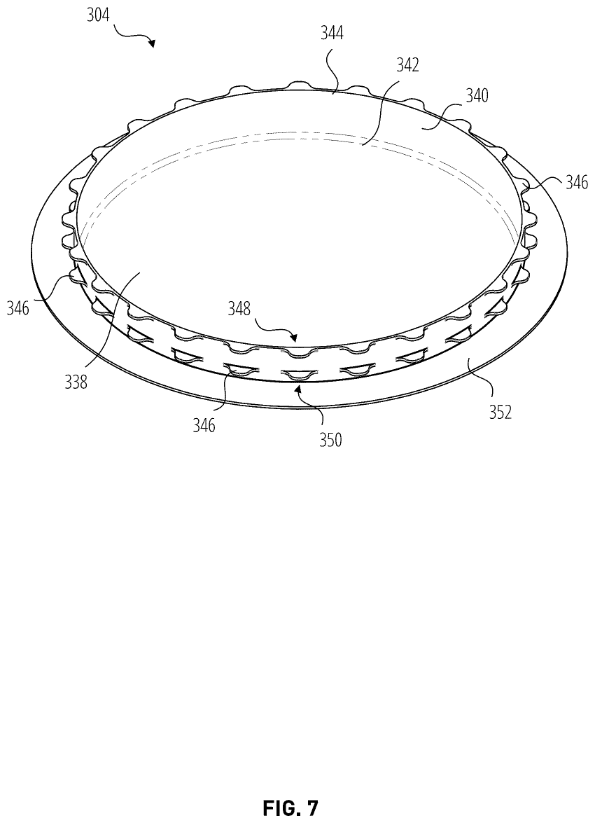

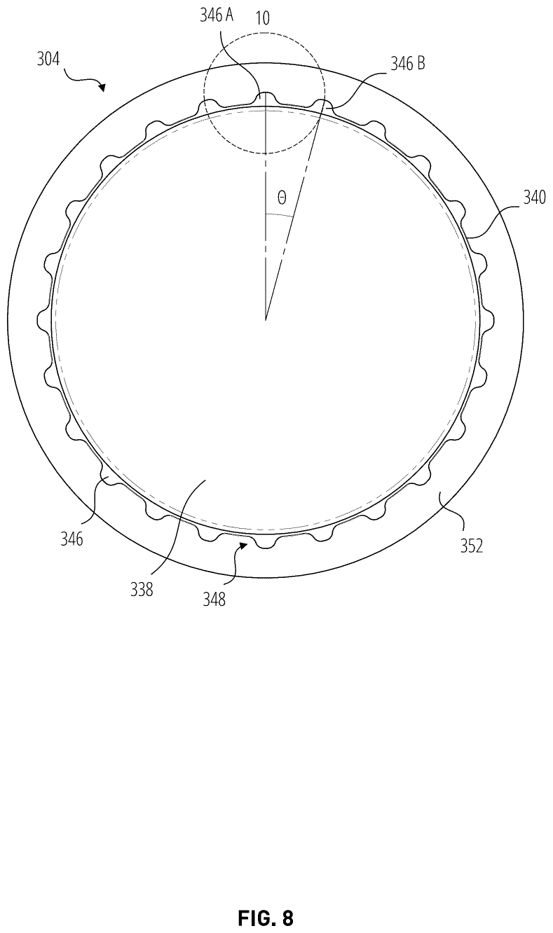

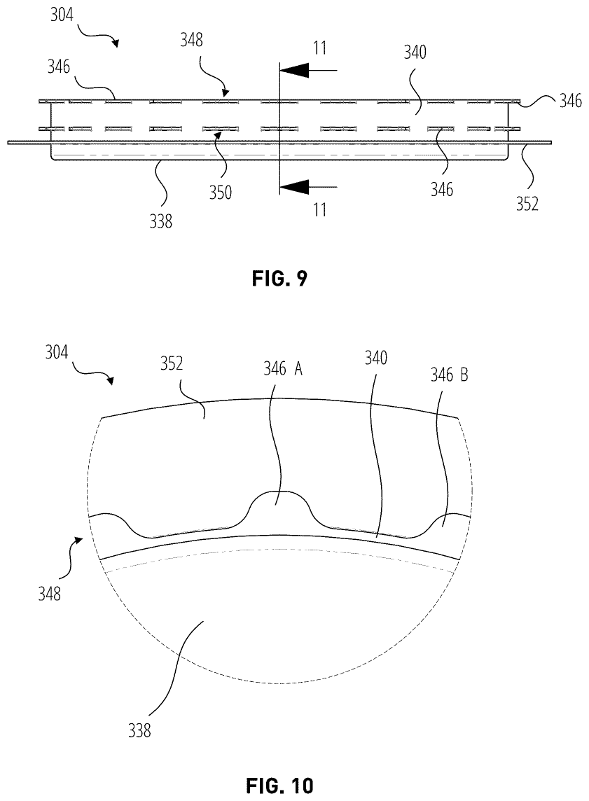

With reference to FIG. 7, FIG. 8, and FIG. 9, provided is a perspective, top, and side view of the first example of the heat conductive body 304, respectively.

The heat conductive body 304 comprises a contacting wall 338. The contacting wall 338 may be configured to contact a heater plate (e.g., heater plate 230 of respiratory therapy system 200), in use. The contacting wall 338 may comprise a circular shape when viewed from above. In some examples, the contacting wall 338 may be a polygon when viewed from above.

The size and/or shape of the heat conductive body 304, e.g., contacting wall 338, may be substantially similar to the heater plate 230 which may improve thermal conduction between the heat conductive body 304 and the heater plate 230. The shape of the heat conductive body 304 may be configured to reduce hotspots and variation of the heat distribution across the heat conductive body in use. The shape of the heat conductive body 304 may be substantially similar to the shape of the heater plate 230.

An upstanding wall 340 extends from the contacting wall 338. The upstanding wall 340 comprises a proximal end 342 at or adjacent the contacting wall 338 and a distal end 344 away from the contacting wall 338. In some examples, the heat conductive body 304 may comprise more than one upstanding wall 340. The upstanding wall 340 may extend from the periphery of the contacting wall 338. The upstanding wall 340 may be substantially perpendicular with respect to the contacting wall 338. The upstanding wall 340 may be configured to strengthen and/or stiffen the contacting wall 338. The upstanding wall 340 may ameliorate warping or buckling of the contacting wall 338. The size and/or shape of the heat conductive body 304, e.g., upstanding wall 340 may be complementary to the second end 310 of the hollow body 302. The upstanding wall 340 may be configured to protrude into the second end 310 of the hollow body 302 when the humidification chamber 300 is assembled, as best shown in FIG. 6.

The upstanding wall 340 and the contacting wall 338 in combination may define a dish. The dish may be configured to contain a volume of water or other liquids. When the heat conductive body 304 is assembled with the hollow body 302, the humidification chamber 300 may be configured to contain a relatively larger volume of water. That is, the water level may be above the upstanding wall 340, contained at least in part by the side wall 314 of the hollow body 302. The dish may allow a humidification chamber 300 containing a small volume of liquid to be disassembled without necessarily spilling the volume of liquid.

A plurality of retention protrusions 346 extend substantially laterally from the upstanding wall 340. The plurality of retention protrusions 346 may project outwardly from the upstanding wall 340. In some examples, the plurality of retention protrusions 346 may alternatively project inwardly. The plurality of retention protrusions 346 may be spaced equidistantly about the upstanding wall 340. The plurality of retention protrusions 346 may each extend in a radial direction. The plurality of retention protrusions 346 may extend normal to an outer surface of the upstanding wall 340. Each of the plurality of retention protrusions 346 may be symmetric, e.g., have reflective symmetry about a midplane extending perpendicular to the upstanding wall 340. Each of the plurality of retention protrusion 346 may be identical. In other examples, two or more of the plurality of retention protrusions 346 may be dissimilar. Each of the plurality of retention protrusions 346 may be substantially semi-circular or parabolic in shape, as best shown in FIG. 10. In some examples, the plurality of retention protrusions may have one or more of a variety of shapes.

The plurality of retention protrusions 346 may surround a perimeter of the upstanding wall. The plurality of retention protrusions 346 may comprise between about 2 and 96 retention protrusions 346, for example between about 24 and 72 retention protrusions 346, for example about 48 retention protrusions 346. The plurality of retention protrusions 346 may comprise an upper protrusion array 348 and a lower protrusion array 350. In some examples, the terms “upper” and “lower” may be replaced by “first” and “second” respectively. The lower protrusion array 350 is closer to the contacting wall 338 than the upper protrusion array 348. The upper protrusion array 348 and the lower protrusion array 350 may define a channel configured to receive the sealing element 306. The upper protrusion array 348 and the lower protrusion array 350 may be identical to each other. In some examples, the upper protrusion array 348 and the lower protrusion array 350 may differ from each other. In some examples, the plurality of retention protrusions may comprise an upper protrusion array and a lower singular protrusion. In other examples, the plurality of retention protrusions may comprise a lower protrusion array and an upper singular protrusion. The lower singular protrusion or the upper singular protrusion may be continuous, extending around the entire perimeter of the upstanding wall, or may include a discontinuity. Each of the upper protrusion array 348 and the lower protrusion array 350 may comprise an equal number of retention protrusions 346. Each of the upper protrusion array 348 and the lower protrusion array 350 may comprise between about 12 and 36 retention protrusions 346, for example about 24 retention protrusions 346. Corresponding retention protrusions 346 in the upper protrusion array 348 and the lower protrusion array 350 may be vertically aligned. In other examples, the upper protrusion array 348 may be rotationally, or otherwise, offset from the lower protrusion array 350. In some examples, the retention protrusions of the lower protrusion array 350 may extend further from the upstanding wall 340 than the upper protrusion array 348.

A heat conductive body flange 352 may extend outwardly beyond the upstanding wall 340. As best shown in FIG. 9, the heat conductive body flange 352 may be offset from the contacting wall 338 and project from the upstanding wall 340. The heat conductive body flange 352 may form an annulus about the upstanding wall. The heat conductive body flange 352 may be in the same plane or in a plane which is substantially parallel with the contacting wall 338. The heat conductive body flange 352 may extend outwardly less than the hollow body flange 334 when the humidification chamber 300 is assembled, as best shown in FIG. 6. The heat conductive body flange 352 may be configured to act as a stop for the hollow body flange 334 (or vice versa) during assembly of the humidification chamber 300. The heat conductive body flange 352 and/or the hollow body flange 334 may be configured to engage a slot or slots in a humidifier 106, 222, e.g., base unit, to secure the humidification chamber 300 in place against the heater plate 230. The heat conductive body flange 352 may be configured to cooperate with a protrusion or protrusions of the humidifier 106, 222 to brace the humidification chamber 300 against an upward force from a sprung heater plate 230, ensuring good thermal contact between the contacting wall 338 and the heater plate 230. In some examples, however, a downward force may be alternatively or additionally applied to an upper surface of the heat conductive body flange 352 and/or the hollow body flange 334, either directly or indirectly.

As best shown in FIG. 6, the upper protrusion array 348 and/or the lower protrusion array 350 may be configured to space the sealing element 306 from the heat conductive body flange 352 and/or the opening 326 at the second end 310 of the hollow body 302, when assembled. As shown, the lower protrusion array 350 may be spaced upwardly from the heat conductive body flange 352. The spacing between the upper protrusion array 348 and the lower protrusion array 350 may be greater than the spacing between the lower protrusion array 350 and the heat conductive body flange 352.

A diameter of the heat conductive body 304, e.g., the heat conductive body flange 352, may be between about 80 millimeters (mm) and 150 mm, for example about 115 mm. A height of the upstanding wall 340 may be between about 5 mm and 20 mm, for example about 13 mm.

An underside (i.e., exterior surface) of the contacting wall 338 may be flat or convex (i.e., outwardly curved from the underside of the contacting wall 338). This may improve the heat transfer between a heater plate 230 and the heat conductive body 304, in use. In some examples, the underside of the contacting wall may have a variation of less than about 0.4 mm with respect to a nominal plane. A convex curvature in the contacting wall 338 may be configured so that the contacting wall 338 becomes substantially planar when the humidification chamber 300 is subjected to a biasing force against the heater plate 230 upon engagement with a humidifier 106, 222.

### Sealing Element

The sealing element 306 is configured to provide a seal between the hollow body 302 and the heat conductive body 304, when the humidification chamber 300 is assembled. The seal may be both watertight and airtight, in use.

The plurality of retention protrusions 346 are configured to retain the sealing element 306 in position about the upstanding wall 340 of the heat conductive body 242. The sealing element 306 may be spaced from the heat conductive body flange 352 and/or the opening 326 at the second end 310 of the hollow body 302, when assembled, as best shown in FIG. 6.

The sealing element 306 may be configured to be disposed in a channel between the upper protrusion array 348 and the lower protrusion array 350, when the humidification chamber 300 is assembled. The upper protrusion array 348 may be a first distance from the distal end 344 of the upstanding wall 340. In some examples, the upper protrusion array 348 may be provided at the distal end 344 of the upstanding wall 340 as best shown in FIG. 9. That is, the first distance may be zero. In some examples, an upper surface of the retention protrusions 346 in the upper protrusion array 348 may be flush with an upper surface of the upstanding wall 340, as best shown in FIG. 9. The lower protrusion array 350 may be a second distance from the distal end 344 of the upstanding wall 340. The second distance is greater than the first distance. The difference between the first distance and the second distance may be substantially similar to a height, e.g., diameter, of the sealing element 306. The difference between the first distance and the second distance may be configured to inhibit rolling and/or deflection of the sealing element 306, e.g., as the humidification chamber 300 is assembled.

The sealing element 306 may be an O-ring or a gasket. The sealing element 306 may have a toroidal shape. The sealing element 306 of the first example is shown in the un-deformed state in the drawings. In practice, the hollow body 302, heat conductive body 304 and/or sealing element 306 may be configured so that the sealing element 306 is deformed, e.g., compressed, when the humidification chamber 300 is assembled. The sealing element 306 may have an un-deformed inner diameter (e.g., prior to assembly) substantially similar to an outer diameter of the upstanding wall 340. In some examples, the sealing element 306 may be configured to have an un-deformed internal diameter slightly smaller than the outer diameter of the upstanding wall 340. The sealing element 306 may be resilient. The sealing element 306 may be stretched about the upstanding wall 340, e.g., over the upper protrusion array 348, upon assembly. The sealing element 306 may have a height substantially similar to the distance between the upper protrusion array 348 and the lower upper protrusion array 348. The sealing element 306 may have a height which is about 30% to 80% of a height of the upstanding wall 340, such as about 45%. The sealing element 306 may extend outwardly beyond the retention protrusions 346 towards the hollow body 302 when the humidification chamber 300 is assembled. In some examples, about 30% to 60% of the sealing element 306 may extend beyond the retention protrusions 346, prior to compression or assembly with the hollow body 302.

The sealing element 306 may be formed from a second polymer. The second polymer may comprise a silicone. The dimensions and/or material properties of the sealing element 306 may enable it to form a seal (e.g., a hermetic seal and/or a watertight seal) between the hollow body 302 and the heat conductive body 304. The seal between the hollow body and the heat conductive body is configured to reduce the likelihood of egress of fluid from the humidification chamber 300 when filled with water and/or pressurized, in use.

### Retention Protrusions

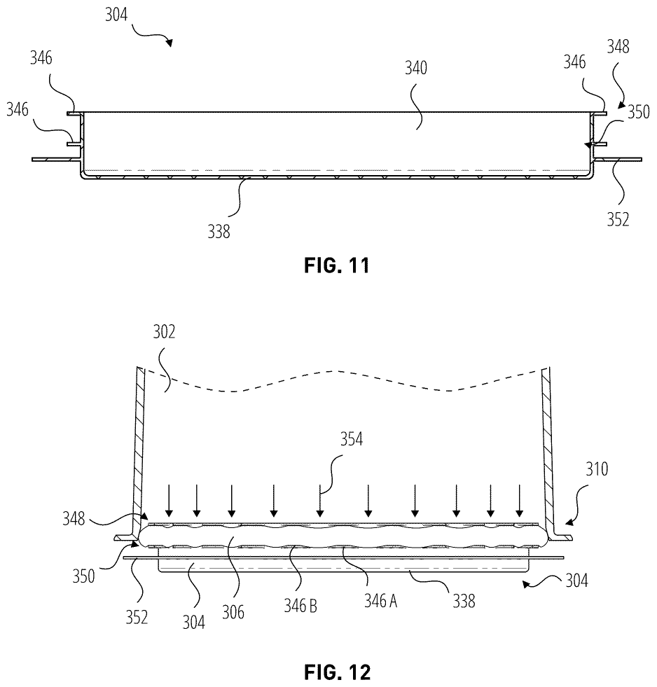

With reference to FIG. 10 and FIG. 11, provided is a detailed top view and side cross-section, respectively, of the first example of the heat conductive body 304 as indicated within FIG. 8 and FIG. 9. A first angle 0 (as shown in FIG. 8) between centers of a retention protrusion 346A and an adjacent retention protrusion 346B may be about 15 degrees. A distance between a retention protrusion 346A and a laterally adjacent retention protrusion 346B may be configured to enable a user to remove the sealing element 306 from the heat conductive body 242. The retention protrusion 346A and laterally adjacent retention protrusion 346B may be any two neighboring retention protrusions identified as a subset of either the upper protrusion array or the lower protrusion array. In some examples, the distance between a retention protrusion 346A and a laterally adjacent retention protrusion 346B may be greater than the width of a typical adult user's thumb or finger. In some examples, a user may remove the sealing element from the heat conductive body by grasping the sealing element and stretching it over the retention protrusions, e.g., of the upper protrusion array 348.

With reference to FIG. 12, provided is a cutaway illustration of potential displacement (e.g., axial displacement) of a sealing element 306 during assembly of the hollow body 302 to the heat conductive body 304. The frictional force between the sealing element 306 and the hollow body 302 may cause the sealing element 306 to be pushed downward and out from its location between the upper protrusion array 348 and the lower protrusion array 350. If the sealing element 306 becomes dislodged during assembly, this may compromise the seal between the heat conductive body 304 and the hollow body 302. For example, downwards movement of the hollow body 302 with respect to the heat conductive body 304 may cause an O-ring sealing element 306 to tend to “roll” down the upstanding wall 340. The displacement of the sealing element 306 is exaggerated in FIG. 12 for illustrative purposes. As the humidification chamber 300 is assembled, the sealing element 306 contacts the hollow body 302 at or near the second end 310, and the resultant force may cause displacement of the sealing element 306 at one or more locations 354 between the plurality of retention protrusions 346. The distance between a first retention protrusion 346A and a laterally adjacent second retention protrusion 346B may be configured to reduce the likelihood or magnitude of the sealing element 306 buckling during assembly and/or disassembly of the humidification chamber 300. In some examples, the distance between the first retention protrusion 346A and the adjacent second retention protrusion 346B may inhibit displacement of the sealing element 306 by increasing the contact surface area between the heat conductive body 304 and the sealing element 306.

The upper protrusion array 348 may be substantially aligned (e.g., vertically aligned) with the lower protrusion array 350. In some examples, however, the upper protrusion array 348 may be rotationally offset with respect to the lower protrusion array 350.

### Thermal Capacity

The mass of the heat conductive body 304 may be less than about 100 grams (g), less than about 50 g, less than about 25 g, or about 23 g. The mass of the heat conductive body 304 may be between about 10 g and 100 g, between about 15 g and 50 g, between about 20 g and 25 g, or about 23 g. In some examples, the mass of the heat conductive body 304 being less than 25 g may improve the performance of the humidification chamber 300 in use.

A thickness of the heat conductive body 304, e.g., at least the contacting wall 338, may be between about 0.2 mm and 1.2 mm, between about 0.2 mm and 1 mm, between about 0.4 mm and 0.9 mm, between about 0.6 mm and 0.8 mm, or about 0.7 mm. In one example, the thickness may be about 0.8 mm. In some examples, the thickness of the heat conductive body 304 may be between about 0.65 mm and 0.75 mm, e.g., about 0.70 mm.

The heat conductive body 304 may be formed from a metal or alloy. The metal or alloy may comprise aluminum or stainless steel. The heat conductive body 304 may be formed in one piece from the metal or alloy. In some examples, e.g., those including discrete retention protrusions 346, the heat conductive body 304 may be milled. In other examples, e.g., those omitting retention protrusions 346, the heat conductive body 304 may be stamped from a sheet of metal or alloy.

The thermal conductivity of the metal or alloy may be between about 12 watts per meter-kelvin (W/mK) and 286 W/mK, between about 88 W/mK and 251 W/mk, between about 170 W/mK and 230 W/mK, e.g., about 227 W/mK, or between about 190 W/mK and 210 W/mK.

A surface of the heat conductive body 304 may comprise a surface coating. In some examples, the heat conductive body 242 may be anodized.

The humidification chamber 300 may be configured to engage a respiratory apparatus such as humidifier 106, 222. A vertical distance between an upper surface of the hollow body flange 334 and an underside of the contacting wall 338, when assembled, may be configured so that the humidification chamber 300 may be securely received and retained by the respiratory apparatus. This distance may ensure consistent contact between a heater plate 230 and the contacting wall 338. Consistent contact between a heater plate and the contacting wall may improve the heat transfer between a heater plate 230 and the heat conductive body 304, in use.

The humidification chamber 300 may be configured to be functionally equivalent to the alternative humidification chamber. In particular, the reusable humidification chamber 300 may be configured so that one or more functions of the humidifier 106, 222, e.g., the base unit 110, 228, continue to operate as normal (e.g., without false alarms) when the humidification chamber 300 is retrofitted to the humidifier 106, 222 in place of an alternative humidification chamber. In some examples, the one or more functions may continue to operate as normal without the need for adjusting a parameter or algorithm of the humidifier (e.g., upon automatic detection or manual selection of the appropriate model of humidification chamber). The humidification chamber 300 and the alternative humidification chamber may differ structurally. For example, the alternative humidification chamber may comprise a heat conductive body permanently attached, e.g., crimped, to a hollow body.

The humidification chamber 300 may be configured to provide a similar level of humidity to the medical gas when compared to the alternative humidification chamber with the same input conditions. The input conditions may include an equivalent steady state heater plate power, volume of water, input gas conditions (i.e., temperature and/or humidity), and ambient temperature. Matching one or more characteristics of the humidification chamber 300 and the alternative humidification chamber may reduce the variation in humidity delivery.

In some examples, the humidification chamber 300 may be configured to have a thermal capacity within about ±15%, within about ±10%, within about ±5%, or within about ±2.5% of a thermal capacity of an alternative humidification chamber, e.g., a disposable humidification chamber, useable with the same humidifier 106, 222. In some examples, the heat conductive body 304 of the humidification chamber 300 may be configured to have a thermal capacity within about ±5 J/K, within about ±2 J/K, within about ±1 J/K, or within about ±0.5 J/K of the thermal capacity of the heat conductive body of the alternative humidification chamber. In some examples, the heat conductive body 304 of the humidification chamber 300 may be configured to have a thermal capacity within about ±15%, within about ±10%, within about 5%, or within about 2.5% of the thermal capacity of the heat conductive body of the alternative humidification chamber.

The thermal capacity of the humidification chamber 300 may be a product of the mass and the specific heat capacity of the humidification chamber 300. In some examples, the specific heat capacity of the heat conductive body 304 may be between about 0.5 joules per gram kelvin (J/gK) and 1.5 J/gK, between about 0.7 J/gK and 1.1 J/gK, between about 0.8 J/gK and 1 J/gK, or about 0.9 J/gK.

The thermal capacity of a humidification chamber 300 may be largely dependent on the thermal capacity of the heat conductive body 304. In some examples, the thermal capacity of the heat conductive body 304 may be less than about 30 joules per kelvin (J/K), less than about 27 J/K, between about 16 J/K and 27 J/K, between about 19 J/K and 24 J/K, between about 20 J/K and 22 J/K, about 21 J/K, or between about 20.7 J/K and 21.2 J/K.

The alternative humidification chamber may be a disposable humidification chamber. The disposable humidification chamber may comprise a hollow body and a heat conductive body which are inseparable (that is, not intended to be removably and repeatedly attached to each other). For example, the heat conductive body of the disposable humidification chamber may be crimped to the hollow body. The disposable humidification chamber may be an F&P™ MR325™ Chamber available from Fisher & Paykel Healthcare Limited of Auckland, New Zealand. The humidifier 106, 222 may be an F&P™ 820™ Heated Humidifier also available from Fisher & Paykel Healthcare Limited, for example. The parameter may be, or may be related to, a particular model of humidification chamber or a property thereof, e.g., thermal capacity. The function may be a low water or water-out alarm. The algorithm may be a low water or water-out detection algorithm. The water-out detection algorithm may involve detecting a response of a humidification chamber to a characteristic energization signal.

The humidification chamber 300 may be configured to withstand at least 20 reprocessing cycles, for example at least 50 reprocessing cycles. In some examples, a reprocessing cycle may comprise placing a humidification chamber 300, for example in the disassembled configuration, in an autoclave. The autoclave may be operated at a temperature of up to about 136° C. for a period of at least 4 minutes. In some examples, a reprocessing cycle may comprise chemical disinfection. In some examples, the humidification chamber 300 may be configured to withstand both autoclaving and chemical disinfection.

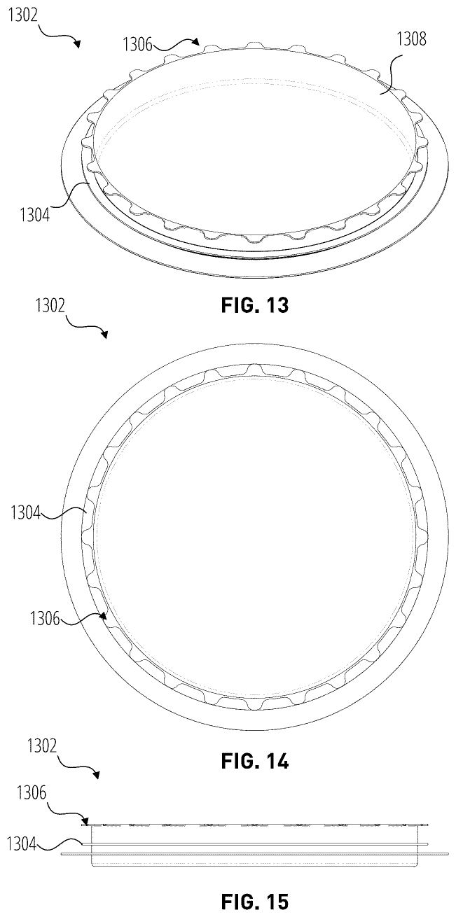

Further examples of heat conductive bodies are illustrated in FIG. 13-FIG. 32.

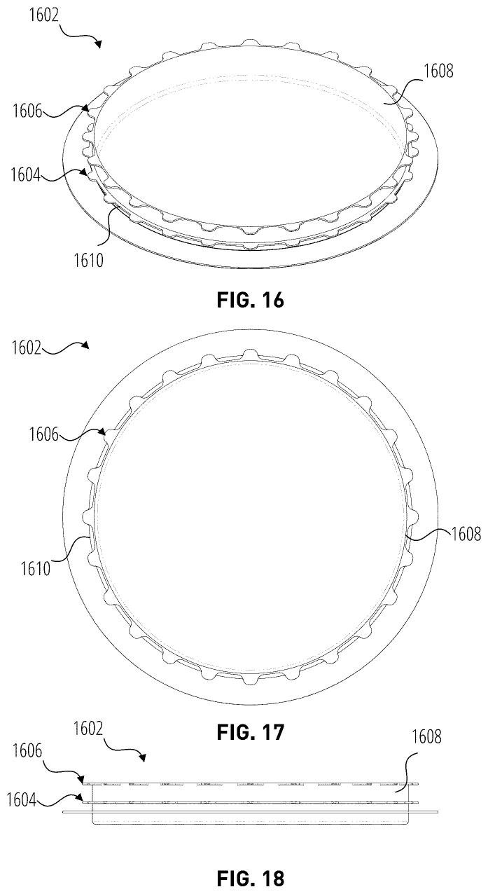

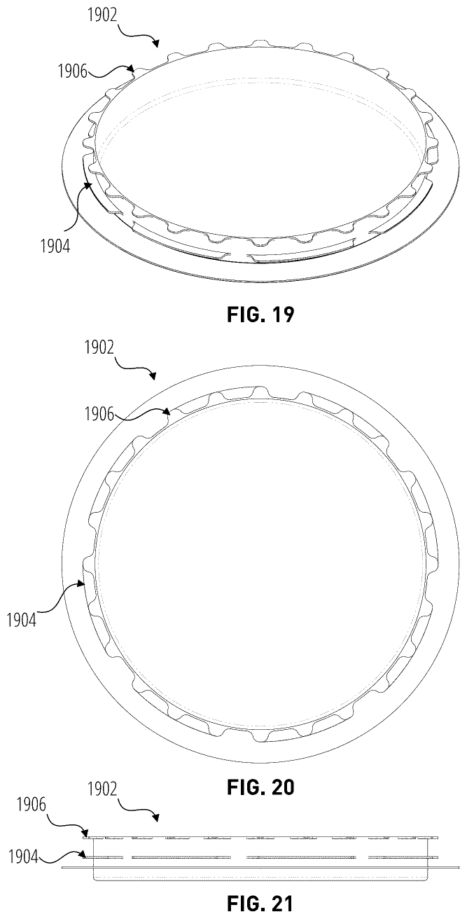

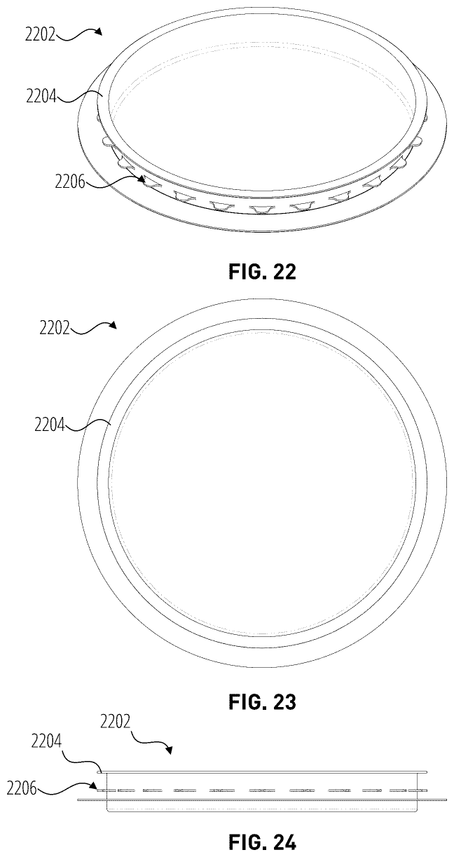

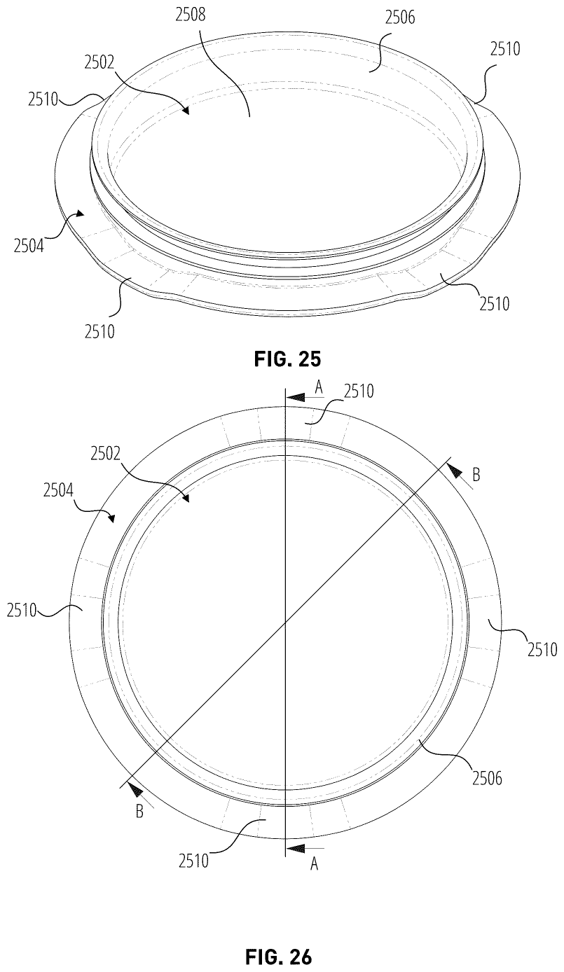

FIG. 13 to FIG. 15 show a second example of a heat conductive body 1302. FIG. 16 to FIG. 18 show a third example of a heat conductive body 1602. FIG. 19 to FIG. 21 show a fourth example of a heat conductive body 1902. FIG. 22 to FIG. 24 show a fifth example of a heat conductive body 2202. And FIG. 25 to FIG. 32 show a sixth example of a heat conductive body 2502.

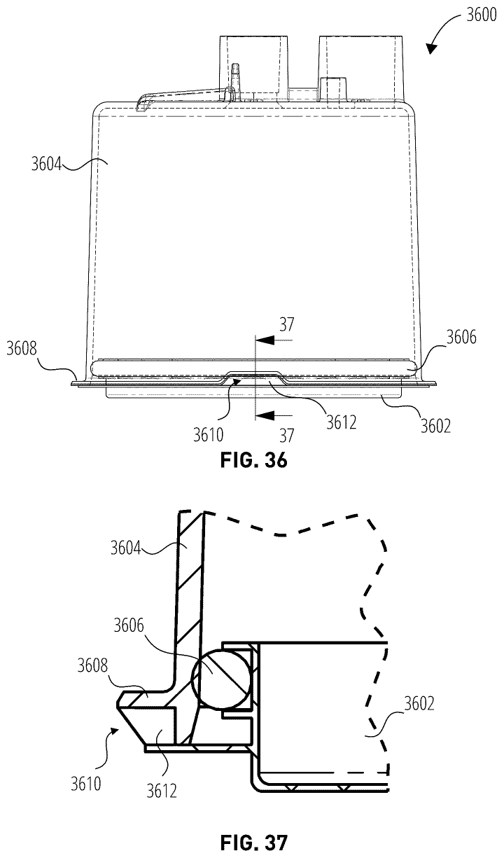

With suitable modifications where necessary, any one of the second to sixth example heat conductive bodies 1302, 1602, 1902, 2202, 2502 may be substituted for the heat conductive body 304 of the first example humidification chamber 300 or the heat conductive body 3602 of the second example humidification chamber described below with reference to FIG. 36 and FIG. 37.

Aside from the differences described below, the second to sixth examples of heat conductive bodies 1302, 1602, 1902, 2202 may be similar to the heat conductive body 304 of the first example humidification chamber 300. In particular, and without limitation, the heat conductive bodies 1302, 1602, 1902, 2202 may have any one or more of the same material, thickness, thermal conductivity, specific heat capacity, mass, or thermal capacity properties as described above with respect to the heat conductive body 304.

The plurality of retention protrusions may be arranged as a protrusion array and a singular protrusion. The retention protrusion(s) at an upper location on the heat conductive body may differ in shape/form to the retention protrusion(s) at a relatively lower location on the heat conductive body. The difference in form between an upper protrusion and a lower protrusion may act to inhibit the displacement of the sealing element as previously described. The form of the retention protrusion at a lower location may be configured to inhibit rolling and/or deflection of the sealing element during assembly of the humidification chamber. The form of the retention protrusion at a lower location may inhibit movement of the sealing element and prevent the seal from being compromised. The dimensions of the retention protrusion(s) at a lower location may be configured to provide a larger contact patch between the sealing element and the lower retention protrusion(s) than between the sealing element and the upper retention protrusion(s). A larger contact patch may provide a greater surface area which acts to retain the seal when a force is applied during assembly and/or disassembly of the humidification chamber.

In the second example heat conductive body 1302 of FIG. 13-FIG. 15, a lower singular retention protrusion 1304 is provided in combination with an upper protrusion array 1306. The lower singular retention protrusion 1304 may be continuous, extending around the entire perimeter of the upstanding wall 1308. The lower singular retention protrusion 1304 may act to retain the sealing element during assembly of the hollow body 302 onto the heat conductive body 1302.

In the third example heat conductive body 1602 of FIG. 16-FIG. 18, the retention protrusions of the lower protrusion array 1604 differ from those of the upper protrusion array 1606. The upper protrusion array 1606 extends directly from the upstanding wall 1608, while the lower protrusion array 1604 extends from a flange member 1610 located on the upstanding wall. The flange member 1610 may be a continuous circumferential flange.

In the fourth example heat conductive body 1902 of FIG. 19-FIG. 21, the retention protrusions of the lower protrusion array 1904 again differ from those of the upper protrusion array 1906. The lower protrusion array 1904 has fewer retention protrusions than the upper protrusion array 1906. The retention protrusions of the lower protrusion array 1904 are longer (in the circumferential direction) than the retention protrusions of the upper protrusion array 1906. The retention protrusions of the lower protrusion array 1904 may span a plurality, e.g., three, of the retention protrusions of the upper protrusion array 1906.

In further examples, the upper and lower retention protrusion(s) of the second example, third example, or fourth example heat conductive bodies 1302, 1602, 1902 may be substituted with each other. For example, in the fifth example heat conductive body 2202 of FIG. 22-FIG. 24, an upper singular protrusion 2204 is provided in combination with a lower protrusion array 2206. The retention protrusions in this example may be configured to improve the retention and/or reduce deformation of the sealing element during disassembly of the humidification chamber.

In some examples, the humidification chamber may comprise a surround, e.g., instead of, or in addition to, a heat conductive body flange and/or retention protrusions of the heat conductive body. The surround may be integral with, attached to, or attachable with the heat conductive body, e.g., the upstanding wall. The surround may be configured to engage the base unit of a humidifier, e.g., to guide, brace, and/or retain the humidification chamber. The combination of the heat conductive body 2502 and the surround 2504 may be referred to as the base of the humidification chamber.

In some examples, the humidification chamber may comprise one or more removal features e.g., in one or more of the hollow body, the heat conductive body, and/or the surround.

In the sixth example heat conductive body 2502 of FIG. 25-FIG. 32, the heat conductive body 2502, e.g., the upstanding wall 2506, is attached to a surround 2504. The surround 2504 may partially or completely surround (e.g., encircle) the heat conductive body 2502, e.g., the upstanding wall 2506. With reference to FIG. 25 and FIG. 26, provided are perspective and top views, respectively, of the heat conductive body 2502 and the surround 2504.

The heat conductive body 2502 and the surround 2504 may be used with the hollow body 302 of the humidification chamber 300, for example. The surround 2504 may provide one or more similar features or functions to the heat conductive body flange and/or the retention protrusions previously described.

The heat conductive body 2502 may comprise a contacting wall 2508 and an upstanding wall 2506 extending from the contacting wall 2508 as described with respect to the previous examples. In some examples, the heat conductive body 2502 does not extend (e.g., in the radial direction) beyond the surround 2504 and/or the upstanding wall 2506. In some examples, the heat conductive body 2502 may comprise an upper protrusion array or an upper singular protrusion as previously described. In some examples, a sealing element (not shown) may be located adjacent to at least one of the heat conductive body 2502 and the surround 2504. The channel may be formed between the heat conductive body 2502 and the surround 2504.

The surround 2504 may be formed in a separate step to the heat conductive body 2502. The surround 2504 may be connected to the heat conductive body 2502, e.g., the upstanding wall 2506, by interference fit, adhesive, or overmolding, for example. The surround 2504 may be permanently connected to the heat conductive body, e.g., not configured to be non-destructively separable.

The heat conductive body 2502 may be formed from a metal or alloy. The metal or alloy may be aluminum or stainless steel, e.g., grade 304 stainless steel.

The surround 2504 may be formed of a dissimilar material to the heat conductive body 2502. The surround 2504 may be a formed from a polymer. The polymer for the surround 2504 may be selected to withstand a reprocessing cycle, or a predetermined minimum number of reprocessing cycles, without distortion and/or degradation due to heat and/or chemicals. The polymer may comprise polypropylene. In some examples, the surround 2504 and the heat conductive body 2502 in combination may be configured to withstand reprocessing as described above with respect to the humidification chamber 300. The surround may have a relatively lower thermal conductivity than the heat conductive body.

The surround 2504 being formed from a dissimilar material may provide for a reduced, e.g., negligible, contribution to the thermal capacity of the humidification chamber.

The surround 2504 may simplify manufacture and/or reduce manufacturing costs for the heat conductive body 2502.

In some examples, the heat conductive body 2502, or at least the contacting wall 2508, may have a uniform thickness. The heat conductive body 2502, or the contacting wall 2508, may be between about 0.2 mm and 1.2 mm, between about 0.2 mm and 1 mm, between about 0.4 mm and 0.9 mm, between about 0.5 mm and 0.9 mm thick, or between about 0.6 mm and 0.8 mm thick, e.g., about 0.7 mm thick. In another example, the contacting wall 2508 may be about 0.8 mm thick. The heat conductive body 2502 may be formed from a sheet material, e.g., a sheet of aluminum alloy. The heat conductive body 2502 may be stamped. The heat conductive body 2502 may be formed in a stamping press.

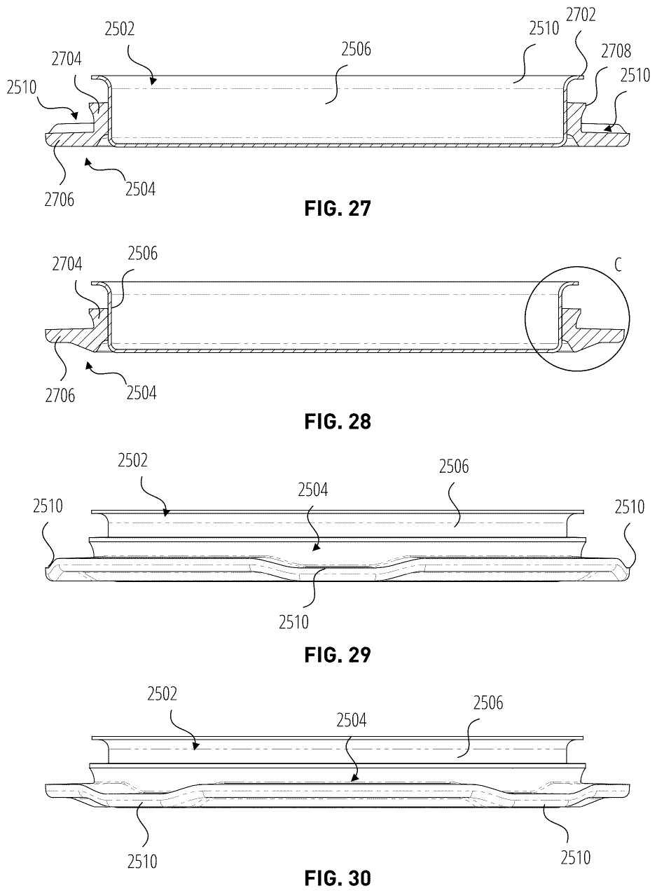

With reference to FIG. 27 and FIG. 28, provided are cross-section views of the sixth example heat conductive body 2502 through planes A-A and B-B, respectively, as indicated in FIG. 26.

The heat conductive body 2502 may have a lip 2702. The lip 2702, in cross-section, may curve outwardly from the distal end of the upstanding wall 2506. The lip 2702 may form an upper singular protrusion as described above. The lip 2702 may at least in part define the channel configured to receive the sealing element.

The surround 2504 may adjoin the upstanding wall 2506. The surround 2504 may form an annulus about the upstanding wall 2506.

A first portion 2704 of the surround 2504, e.g., an upper surface of the surround 2504 in cross-section, may form a shoulder. The shoulder may at least in part retain the sealing element when the humidification chamber is assembled. The shoulder may at least in part define the channel configured to receive the sealing element. In some examples, the humidification chamber may be configured to retain the sealing element between the shoulder and the lip 2702. In some examples, the first portion 2704 of the surround 2504 and/or the lip 2702 of the heat conductive body 2502 may comprise one or more retention protrusions for the sealing element.

In some examples, a second portion 2706 of the surround 2504, e.g., a lower portion of the surround 2504 in cross-section, may comprise a removal feature 2510 as shown in at least FIG. 25 and FIG. 27. The removal feature 2510 may be configured to aid disassembly of the hollow body from the base of the humidification chamber, i.e., the surround and the heat conductive body 2708. In various examples, the surround 2504 may comprise one or more removal features. In the illustrated example, the surround 2504 comprises four removal features 2510.

The removal feature 2510 may comprises a stepped region, e.g., a downwardly stepped region, which provides a slot between the surround 2504 and the hollow body, e.g., the hollow body flange 334, when the humidification chamber is in the assembled configuration.

In at least some examples, the humidification chamber may be disassembled at least in part by use of an implement (not shown) suitable for insertion into the slot. The implement may be maneuvered (e.g., rotated and/or levered) in the slot to prize the hollow body from the surround 2504 and the heat conductive body 2502 (or vice versa).

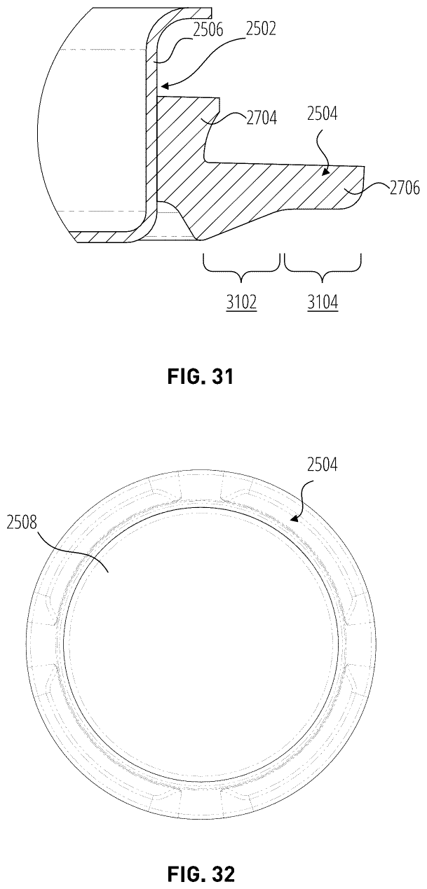

As shown in FIG. 28, in particular detail C shown in FIG. 31, the surround 2504, e.g., second portion 2706, may comprise features previously described in relation to the heat conductive body flange of the preceding examples. For example, the second portion 2706 of the heat conductive body surround 2704 may be configured to act as a stop for the hollow body flange 334 during assembly of the humidification chamber.

With reference to FIG. 29 and FIG. 30, provided are front and perspective views of the heat conductive body 2502 in accordance with the sixth example.

FIG. 31 is a detailed view of a heat conductive body 2502 in accordance with the sixth example, as indicated by detail C in FIG. 28.

In some examples, as best illustrated in FIG. 31, the surround 2504 may be substantially ‘ [symbol] ’-shaped in cross-section. The second portion 2706 may extend in a substantially outward, e.g., radial, direction. The first portion 2704 may extend in a substantially perpendicular direction, e.g., an upwards direction, with respect to the second portion 2706.

At least part of the surround 2504, e.g., at least part of the second portion 2706 intermediate the removal features 2510 as best shown in FIG. 27, may have a substantially uniform thickness. In some examples, the surround 2504 in cross-section may transition from a varying thickness in at least part of the first portion 2704 to a substantially uniform thickness in at least part of the second portion 2706, as best shown in FIG. 31. In some examples, at least part of the second portion 2706, e.g., distal from the first portion 2704, may have a thickness of between about 2.5 mm and 3.5 mm, e.g., about 3 mm.

At least part of the surround 2504, e.g., at least part of the second portion 2706 at the removal feature 2510 as best shown in FIG. 31, may at least in part taper in thickness. For example, the second portion 2706 may taper outwardly from adjacent the first portion 2704. The second portion 2706 may have a tapered section 3102 and a uniform section 3104. In some examples, as shown in FIG. 31, the tapered section 3102 and the uniform section 3104 may each correspond to about half of a length of the second portion 2706.

In some examples, as shown in FIG. 31, an underside of at least part of the second portion 2706, e.g., at a removal feature 2510, may be concave.

A height of the removal feature 2510 may be expressed as the distance between the surround 2504, e.g., second portion 2706, and the hollow body flange at the removal feature 2510 when the humidification chamber is assembled. In some examples, the height of the removal feature, i.e., the height of the slot, may be between about 1 mm and 3 mm, e.g., about 2 mm.

A width of the removal feature 2510 may be expressed with reference to a chord length of the second portion 2706 distal from the first portion 2704. In some examples, the width of the removal feature 2510 may be at least about 5 mm, between about 5 mm and 100 mm, between about 5 mm and 50 mm, between about 5 mm and 30 mm, between about 10 mm and 25 mm, or between about 15 mm and 20 mm, e.g., about 16 mm. In some examples, a humidification chamber may have a single continuous removal feature extending about a majority, or an entirety, of a perimeter of the humidification chamber. In some examples, the width of the removal feature 2510 may be between about 10 mm and 22 mm, or between about 13 mm and 19 mm, e.g., about 16 mm. In some examples, the width of the removal feature may be between about 10 mm and 20 mm, or between about 12 mm and 16 mm, e.g., about 14 mm.

In some examples with more than one removal feature 2510, each removal feature 2510 may have identical geometry. In some examples, each removal feature 2510 may be equidistantly spaced about the perimeter of the surround 2504. In some examples, pairs of removal features 2510 may be diametrically opposed.

A removal feature 2510, e.g., stepped region, being provided in a surround 2504 may be preferable to a removal feature provided in the hollow body, e.g., hollow body flange. In some examples, a removal feature 2510 being a provided in the surround 2504 may enable the engagement of the humidification chamber with the humidifier to be independent of rotational orientation of the humidification chamber with respect to a vertical axis. In some examples, a removal feature, e.g., a stepped region, in a hollow body flange may inhibit engagement of the humidification chamber with at least some humidifiers and/or in some orientations.

FIG. 32 is a bottom view of the heat conductive body 2502 and the surround 2504 in accordance with the sixth example.

In a seventh example, a heat conductive body may be formed from stainless steel. The heat conductive body, e.g., the contacting wall, may have a thickness of between about 0.4 mm and 0.7 mm, between 0.5 and 0.6 mm, or about 0.55 m. The heat conductive body may have a thermal conductivity of between about 10 W/mK and 20 W/mK, between about 14 W/mK and 18 W/mK, or about 16 W/mK. The heat conductive body may have a specific heat capacity of between about 0.3 J/gK and 0.7 J/gK, between about 0.4 J/gK and 0.5 J/gK, or about 0.5 J/gK. The heat conductive body may have a mass of between about 30 g and 40 g, between about 33 g and 37 g, or about 35 g. The heat conductive body may comprise a thermal capacity of between about 15 J/K and 20 J/K, between about 16 J/K and 19 J/K, or about 17 J/K. The heat conductive body optionally may not have an upstanding wall. The heat conductive body may comprise a flat plate. The heat conductive body may be integral with, attached to, or attachable with a surround, as described above.

### Second Example Humidification Chamber

During set up of a respiratory apparatus, a humidification chamber may be mounted onto a humidifier base unit. The mounting process may involve depressing a component of the humidifier 106 and moving the humidification chamber 108 in a direction to be received by the humidifier 106.

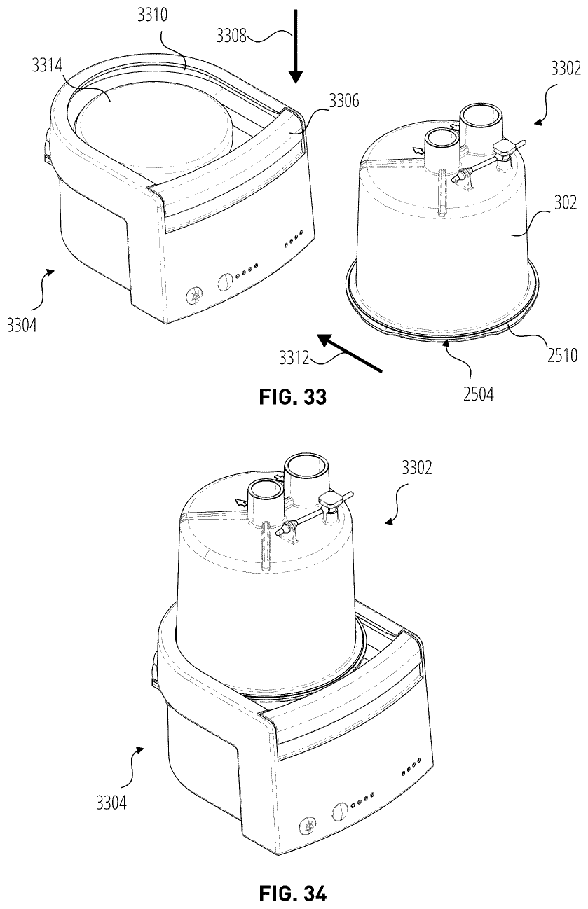

FIG. 33 is a perspective view of second example humidification chamber 3302 as it is being mounted to the base unit of a humidifier 3304. Aside from the differences described below, the humidification chamber 3302 according to this second example may be similar to the humidification chamber 300 of the first example, and the description of the first example is intended to apply also to the humidification chamber 3302.

The second example humidification chamber 3302 may comprise the hollow body 302 of the first example humidification chamber 300 of FIG. 3. The base of the humidification chamber 3302 may comprise the combination of the sixth example heat conductive body 2502 and the surround 2504 of FIG. 25.

The humidifier 3304 may be an F&P™ 820™ heated humidifier.

The humidifier 3304 may have a guard 3306. The guard 3306 may be first depressed by a user in the direction indicated by the arrow 3308. Depressing the guard 3306 may expose a shoulder 3310 in the humidifier 3304 which may be configured to receive the hollow body flange and/or the surround of the humidification chamber 3302. The humidification chamber 3302 may be moved in direction indicated by the arrow 3312, e.g., a horizontal direction, to be received by the humidifier 3304. Engagement of the hollow body flange and/or the surround with the shoulder 3310 may brace the humidification chamber 3302 against an upward force from an upwardly biased, e.g., sprung, heater plate 3314 of the humidifier 3304. The guard 3306 may resiliently return to a guard position following removal of the depressing force and mounting of the humidification chamber 3302.

FIG. 34 is a perspective view of the humidification chamber 3302 mounted on the humidifier 3304.

As illustrated in FIG. 34, with the humidification chamber 3302 mounted to the humidifier 3304, the guard 3306 in the guard position may be configured to impede finger access to the heater plate 3314, which may become hot during use, and/or impede inadvertent removal of the humidification chamber 3302 from the humidifier 3304.

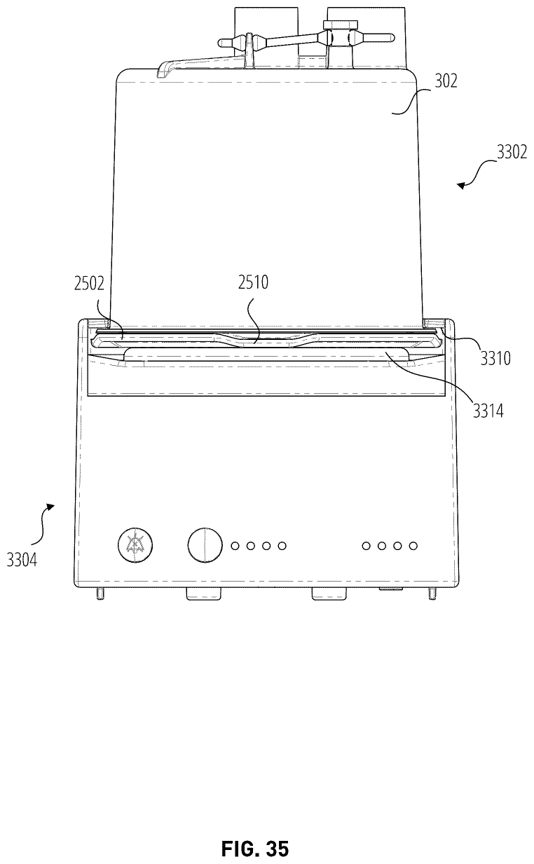

FIG. 35 is a front view of a humidification chamber 3302 mounted on the humidifier 3304. The guard 3306 is omitted in FIG. 35 for illustrative purposes.

When the humidification chamber 3302 is mounted on the humidifier 3304, an upper surface of the hollow body flange may engage the shoulder 3310 of the humidifier 3304. And the contacting wall may engage the heater plate 3314 of the humidifier 3304.

A vertical distance between the shoulder 3310 of the humidifier 3304 and the contacting wall may help secure the humidification chamber 3302 when mounted on the humidifier 3304. The shape of the surround 2504 may be configured to ease the process of mounting the humidification chamber 3302 onto the humidifier 3304. The shape of the surround 2504 may be configured to ease the process of removing the humidification chamber 3302 from the humidifier 3304.

### Third Example Humidification Chamber

With reference to FIG. 36 and FIG. 37, provided is a front view and a detailed cross-section of a third example of a humidification chamber 3600. The humidification chamber 3600 broadly comprises a hollow body 3604, a heat conductive body 3602 and a sealing element 3606. Aside from the differences described below, the humidification chamber 3600 according to this third example may be similar to the humidification chamber 300 of the first example, and the description of the first example is intended to apply also to the humidification chamber 3600. Alternatively, the heat conductive body 3602 may be similar to any one of the second to fifth heat conductive bodies 1302, 1602, 1902, 2202.

The hollow body 3604 has a hollow body flange 3608. The hollow body flange 3608 may comprise one or more removal features 3610 3610. The removal feature 3610 comprises a stepped region, e.g., an upwardly stepped region, which provides a slot 3612 between the heat conductive body 3602 and the hollow body 3604 when the humidification chamber 3600 is in the assembled configuration. The slot 3612 is configured such that the heat conductive body 3602 may be disassembled from the hollow body 3604 by an implement (not shown) configured to be inserted into the slot 3612. The implement may be maneuvered (e.g., rotated and/or levered) in the slot 3612 to prize the hollow body 3604 from the heat conductive body 3602. In some examples, the humidification chamber 3600 may comprise more than one removal feature 3610. In some examples, the implement may be retained on the humidification chamber 300 by a tether. In some examples, the implement may be retained on the humidification chamber 300 by the refill port tether 332. In other examples, the heat conductive body 3602 may alternatively or additionally comprise a stepped region.

The examples described above are to be considered illustrative rather than restrictive. The humidification chambers 300, 3302, 3600 may be modified in numerous ways, some of which have been described above.

Unless the context clearly requires otherwise, throughout the description and claims the words “comprising,” “including” and variants (e.g., “comprise” and “comprises”) are used in an inclusive sense, rather than an exclusive sense. That is, such terms are to be construed in the sense of “including, but not limited to” rather than “consisting solely of.” Any reference to publications or products throughout the specific action should in no way be considered as an admission that the publication or product is necessarily prior art, analogous, widely known or forms part of common general knowledge in the field.

Where reference is made throughout the description and claims to particular orientations or directions (e.g., “upper,” “lower,” “upwardly,” “down,” “downwards,” “top,” “base,” “vertical” and the like), those terms are used in a relative rather than absolute sense, and with reference to the example respiratory apparatus 100 and respiratory therapy system 200 in which a humidification chamber is configured to sit atop a heater plate in the orientation illustrated in the accompanying drawings. However, alternative configurations are contemplated without departing from the spirit or scope of the disclosure.

### LISTING OF DRAWING ELEMENTS

100 respiratory apparatus
102 gases source
104 flow generator
106 humidifier
108 humidification chamber
110 base unit
112 inlet port
114 humidifier supply tube
116 outlet port
118 inspiratory tube
120 tube connector
122 electrical connector
200 respiratory therapy system
202 patient
204 patient interface
206 inspiratory tube
208 ventilator
210 gases inlet
212 flow source
214 ventilator user interface
216 ventilator controller
218 ventilator sensor
220 gases source
222 humidifier
224 humidifier supply tube
226 humidification chamber
228 base unit
230 heater plate
232 humidifier user interface
234 humidifier controller
236 electrical lead
238 humidifier sensor
240 heater wire
242 heat conductive body
300 humidification chamber
302 hollow body
304 heat conductive body
306 sealing element
308 first end
310 second end
312 top wall
314 side wall
316 inlet port
318 outlet port
320 baffle
322 indicia
324 chamber rib
326 opening
328 refill port
330 refill port stopper
332 refill port tether
334 hollow body flange
336 chamber
338 contacting wall
340 upstanding wall
342 proximal end
344 distal end
346 retention protrusion
348 upper protrusion array
350 lower protrusion array
352 heat conductive body flange
354 displacement location
1302 heat conductive body
1304 lower singular retention protrusion
1306 upper protrusion array
1308 upstanding wall
1602 heat conductive body
1604 lower protrusion array
1606 upper protrusion array
1608 upstanding wall
1610 flange member
1902 heat conductive body
1904 lower protrusion array
1906 upper protrusion array
2202 heat conductive body
2204 upper singular protrusion
2206 lower protrusion array
2502 heat conductive body
2504 surround
2506 upstanding wall
2508 contacting wall
2510 removal feature
2702 lip
2704 first portion
2706 second portion
2708 retention protrusion
3102 tapered section
3104 uniform section
3302 humidification chamber
3304 humidifier
3306 guard
3308 arrow
3310 shoulder
3312 arrow
3314 heater plate
3600 humidification chamber
3602 heat conductive body
3604 hollow body
3606 sealing element
3608 hollow body flange
3610 removal feature
3612 slot

What is claimed is:

1. A humidification chamber for respiratory or surgical humidification, comprising:
a hollow body, comprising an inlet port and an outlet port;
a heat conductive body; and
a sealing element configured to provide a seal between the hollow body and the heat conductive body, when assembled;
wherein the heat conductive body is configured to be removably attachable to the hollow body and has a thermal capacity of less than about 27 joules per kelvin (J/K), between about 16 J/K and 27 J/K, between about 19 J/K and 24 J/K, between about 20 J/K and 22 J/K, about 21 J/K, or between about 20.7 J/K and 21.2 J/K.

2. The humidification chamber of claim 1, the heat conductive body comprising an aluminum alloy material.

3. The humidification chamber of claim 1 or 2, the heat conductive body comprising a thickness of between about 0.2 mm and 1.2 mm, between about 0.2 mm and 1 mm, between about 0.4 mm and 0.9 mm, between about 0.6 mm and 0.8 mm, or about 0.7 mm.

4. The humidification chamber of any one of claims 1 to 3, the heat conductive body comprising a thermal conductivity of between about 12 W/mK and 286 W/mK, between about 88 W/mK and 251 W/mK, between about 170 W/mK and 230 W/mK, about 227 W/mK, or between about 190 W/mK and 210 W/mK.

5. The humidification chamber of any one of claims 1 to 4, the heat conductive body comprising a specific heat capacity of between about 0.5 J/gK and 1.5 J/gK, between about 0.7 J/gK and 1.1 J/gK, between about 0.8 J/gK and 1 J/gK, or about 0.9 J/gK.

6. The humidification chamber of any one of claims 1 to 5, the heat conductive body comprising a mass of between about 10 g and 100 g, between about 15 g and 50 g, between about 20 g and 25 g, or about 23 g.

7. The humidification chamber of any one of claims 1 to 6, comprising one or more removal features configured to aid disassembly of the humidification chamber.

8. The humidification chamber of any one of claims 1 to 7, the heat conductive body at least in part defining a channel configured to receive and retain the sealing element, in use.

9. The humidification chamber of any one of claims 1 to 8, comprising a surround, the surround integral with, attached to, or attachable with the heat conductive body and configured to engage a base unit of a humidifier, in use.

10. The humidification chamber of claim 9, wherein the surround is overmolded to the heat conductive body.

11. The humidification chamber of claim 9 or 10, the surround comprising one or more removal features configured to aid disassembly of the humidification chamber.

12. The humidification chamber of any one of claims 9 to 11, the surround at least in part defining a channel configured to receive and retain the sealing element, in use.

13. The humidification chamber of any one of claims 1 to 12, the heat conductive body comprising a contacting wall configured to be in thermal contact with a heater plate, in use, the contacting wall comprising a uniform thickness of between about 0.2 mm and 1.2 mm, between about 0.2 mm and 1 mm, between about 0.4 mm and 0.9 mm, between about 0.6 mm and 0.8 mm, or about 0.7 mm.

14. A humidification system for respiratory or surgical humidification, comprising:
a humidifier base unit comprising a heater plate;
a first humidification chamber configured to be engageable with the humidifier base unit in thermal contact with the heater plate; and
a second humidification chamber configured to be engageable with the humidifier base unit in thermal contact with the heater plate;
wherein the first humidification chamber and the second humidification chamber differ structurally, and a thermal capacity of the second humidification chamber is substantially equivalent to a thermal capacity of the first humidification chamber.

15. The humidification system of claim 14, wherein the first humidification chamber comprises a disposable humidification chamber configured to be disassembled and reassembled, and the second humidification chamber comprises a reusable humidification chamber which is permanently assembled.

16. The humidification system of claim 14 or 15, wherein a thermal capacity of the second humidification chamber is within 15%, within 10%, within 5%, or within 2.5% of a thermal capacity of the first humidification chamber.

17. The humidification system of any one of claims 14 to 16, wherein a thermal capacity of the second humidification chamber is less than 30 joules per kelvin (J/K), less than about 27 J/K, between about 16 J/K and 27 J/K, between about 19 J/K and 24 J/K, between about 20 J/K and 22 J/K, about 21 J/K, or between about 20.7 J/K and 21.2 J/K.

18. The humidification system of any one of claims 14 to 17, wherein the second humidification chamber comprises the humidification chamber of any one of claims 1-13 or 27-60.

19. A humidification system for respiratory or surgical humidification, comprising:
a humidifier base unit comprising a heater plate;
a first humidification chamber configured to be engageable with the humidifier base unit in thermal contact with the heater plate; and
a second humidification chamber configured to be engageable with the humidifier base unit in thermal contact with the heater plate;
wherein the first humidification chamber and the second humidification chamber differ structurally, and the second humidification chamber is configured to be substitutable for the first humidification chamber without adjusting a parameter or algorithm of the humidifier base unit.

20. The humidification system of claim 19, wherein the first humidification chamber and the second humidification chamber are configured to provide a similar level of humidity to a medical gas with the same input conditions, in use.

21. The humidification system of claim 19 or 20, wherein the first humidification chamber and the second humidification chamber are configured to provide a similar level of humidity to a medical gas at an equivalent steady state heater plate power, in use.

22. The humidification system of any one of claims 19 to 21, wherein the humidifier base unit is configured to detect a low water or water-out condition.

23. The humidification system of any one of claims 19 to 22, wherein the humidifier base unit is configured to determine a response of the first humidification chamber or the second humidification chamber, plus a volume of water (if any) contained therein, to a characteristic energization signal applied to the heater plate.

24. The humidification system of any one of claims 19 to 23, wherein a thermal capacity of the second humidification chamber is substantially equivalent to a thermal capacity of the first humidification chamber.

25. The humidification system of any one of claims 19 to 24, wherein a thermal capacity of each of the first humidification chamber and the second humidification chamber is between about 16 J/K and 27 J/K, between about 19 J/K and 24 J/K, between about 20 J/K and 22 J/K, or about 21 J/K.

26. The humidification system of any one of claims 19 to 25, the second humidification chamber comprising the humidification chamber of any one of claims 1-13 or 27-60.

27. A humidification chamber for respiratory or surgical humidification, the humidification chamber comprising:
a hollow body, the hollow body comprising an inlet port and an outlet port;
a heat conductive body configured to be removably attached to the hollow body, the heat conductive body comprising a contacting wall; and
a surround, the surround integral with, attached to, or attachable with the heat conductive body, and comprising a removal feature;
wherein the hollow body and the heat conductive body are configured to at least in part define a chamber configured to contain a volume of water, in use.

28. The humidification chamber of claim 27, the removal feature comprising a stepped region configured to form a slot between the surround and the hollow body when the humidification chamber is assembled, in use.

29. The humidification chamber of claim 28, wherein the slot is configured for insertion of an implement.

30. The humidification chamber of any one of claims 27 to 29, comprising a sealing element configured to be located adjacent to the heat conductive body and the surround, the sealing element configured to provide a seal between the hollow body and one or more of the heat conductive body or the surround.

31. The humidification chamber of claim 30, the surround comprising a first portion proximal to the heat conductive body and a second portion distal from the heat conductive portion, the first portion forming a shoulder configured to at least in part retain the sealing element, and the second portion comprising the removal feature.

32. The humidification chamber of claim 31, the second portion at least in part comprising a substantially uniform thickness of between about 2.5 mm and 3.5 mm, e.g., about 3 mm.

33. The humidification chamber of claim 31 or 32, the second portion of the surround configured to act as a stop for the hollow body during assembly of the humidification chamber, in use.

34. The humidification chamber of any one of claims 27 to 33, wherein:
a height of the removal feature is between about 1 mm and 3 mm, e.g., about 2 mm; and/or a width of the removal feature is at least about 5 mm, between about 5 mm and 100 mm, between about 5 mm and 50 mm, between about 5 mm and 30 mm, between about 10 mm and 25 mm, or between about 15 mm and 20 mm, e.g., about 16 mm.

35. The humidification chamber of any one of claims 27 to 34, the removal feature configured to aid disassembly of the hollow body and the heat conductive body.

36. The humidification chamber of any one of claims 27 to 35, the surround comprising more than one removal feature.

37. The humidification chamber of any one of claims 27 to 36, the surround comprising between one and eight, or between two and six, e.g., four, removal features.

38. The humidification chamber of any one of claims 27 to 37, the surround comprising a dissimilar material to the heat conductive body, e.g., the surround comprising a polymer such as polypropylene and the heat conductive body comprising a metal or alloy such as aluminum or stainless steel.

39. The humidification chamber of any one of claims 27 to 38, the surround comprising an overmold to the heat conductive body.

40. The humidification chamber of any one of claims 27 to 39, the surround providing a negligible contribution to the thermal capacity of the humidification chamber.

41. The humidification chamber of any one of claims 27 to 40, wherein inserting and maneuvering (e.g., rotating and/or levering) an implement into the removal feature is configured to aid disassembly of the hollow body and the heat conductive body.

42. The humidification chamber of any one of claims 27 to 41, the heat conductive body and the surround, in combination, configured to be removably attached to the hollow body.

43. The humidification chamber of any one of claims 27 to 42, the heat conductive body configured to be removably attached to the hollow body by the surround.

44. The humidification chamber of any one of claims 27 to 43, wherein the surround is permanently connected to the heat conductive body.

45. The humidification chamber of any one of claims 27 to 44, the heat conductive body comprising an upstanding wall extending from the contacting wall, wherein the surround is connected to the upstanding wall.

46. The humidification chamber of any one of claims 27 to 45, the contacting wall formed from an aluminum alloy material.

47. A humidification chamber for use with a humidifier base unit to humidify a flow of medical gases for respiratory therapy or surgical insufflation, the humidification chamber comprising:
a hollow body comprising:
an inlet port for ingress of the flow of medical gases into the humidification chamber, in use; and
an outlet port for egress of the flow of medical gases from the humidification chamber, in use;
a heat conductive body, the hollow body and the heat conductive body in combination configured to form, at least in part, a chamber configured to receive a volume of liquid with a headspace providing a passage for the flow of medical gases from the inlet port to the outlet port; and
a sealing element configured to provide a seal between the hollow body and the heat conductive body.

48. The humidification chamber of claim 47, the heat conductive body comprising an aluminum alloy material.

49. The humidification chamber of claim 47 or 48, the heat conductive body comprising a thickness of between about 0.2 mm and 1.2 mm, between about 0.2 mm and 1 mm, between about 0.4 mm and 0.9 mm, between about 0.6 mm and 0.8 mm, or about 0.7 mm.

50. The humidification chamber of any one of claims 47 to 49, the heat conductive body comprising a thermal conductivity of between about 12 W/mK and 286 W/mk, between about 88 W/mK and 251 W/mK, between about 170 W/mK and 230 W/mk, about 227 W/mK, or between about 190 W/mK and 210 W/mK.

51. The humidification chamber of any one of claims 47 to 50, the heat conductive body comprising a specific heat capacity of between about 0.5 J/gK and 1.5 J/gk, between about 0.7 J/gK and 1.1 J/gK, between about 0.8 J/gK and 1 J/gK, or about 0.9 J/gK.

52. The humidification chamber of any one of claims 47 to 51, the heat conductive body comprising a mass of between about 10 g and 100 g, between about 15 g and 50 g, between about 20 g and 25 g, or about 23 g.

53. The humidification chamber of any one of claims 47 to 52, the heat conductive body comprising a thermal capacity of less than about 30 J/K, less than about 27 J/K, between about 16 J/K and 27 J/K, between about 19 J/K and 24 J/K, between about 20 J/K and 22 J/K, about 21 J/K, or between about 20.7 J/K and 21.2 J/K.

54. The humidification chamber of any one of claims 47 to 53, at least the hollow body and the heat conductive body configured to be repeatedly assembled with, and disassembled from, each other.

55. The humidification chamber of any one of claims 47 to 54, comprising one or more removal features configured to aid disassembly of the humidification chamber.

56. The humidification chamber of any one of claims 47 to 55 comprising a surround, the surround integral with, attached to, or attachable with the heat conductive body and configured to engage a base unit of the humidifier, in use.

57. The humidification chamber of claim 56, wherein the surround is overmolded to the heat conductive body.

58. The humidification chamber of claim 56 or 57, the surround comprising one or more removal features configured to aid disassembly of the humidification chamber.

59. The humidification chamber of any one of claims 47 to 58, the heat conductive body comprising a contacting wall configured to be in thermal contact with a heater plate of a base unit of the humidifier when the humidification chamber is engaged with the base unit, in use, the contacting wall comprising the thickness of between about 0.2 mm and 1.2 mm, between about 0.2 mm and 1 mm, between about 0.4 mm and 0.9 mm, between about 0.6 mm and 0.8 mm, or about 0.7 mm.

60. The humidification chamber of any one of claims 47 to 59, the heat conductive body at least in part defining a channel configured to receive and retain the sealing element, in use.

61. A humidification circuit kit comprising:
the humidification chamber of any one of claims 1-13 or 27-60;
a humidifier supply tube configured to be coupled with the inlet port; and
an inspiratory tube or an insufflation delivery tube configured to be coupled with the outlet port.

62. A humidifier system configured to humidify a flow of medical gases for respiratory therapy or surgical insufflation, the humidifier system comprising:
the humidification chamber of any one of claims 1-13 or 27-60;
an alternative humidification chamber, the alternative humidification chamber differing structurally from the humidification chamber; and
a base unit comprising a heater plate and a controller, the base unit configured to removably receive either of the humidification chamber and the alternative humidification chamber in thermal contact with the heater plate, the heater plate configured to heat the humidification chamber or the alternative humidification chamber to heat and humidify the flow of medical gases passing through the humidification chamber or the alternative humidification chamber, in use, and the controller configured to control the heater plate and to detect one or more of a low water condition or a water-out condition without identification of the humidification chamber or the alternative humidification chamber.

63. The humidifier system of claim 62, the humidification chamber comprising a reusable humidification chamber and the alternative humidification chamber comprising a disposable humidification chamber.

64. A humidifier system configured to humidify a flow of medical gases for respiratory therapy or surgical insufflation, the humidifier system comprising:
a base unit comprising a heater plate configured to generate heat, in use;
a disposable humidification chamber configured to be removably received by the base unit in thermal contact with the heater plate; and
a reusable humidification chamber configured to be removably received by the base unit in thermal contact with the heater plate,
wherein each of the disposable humidification chamber and the reusable humidification chamber comprise a heat conductive body with a thermal capacity between about 16 J/K and 27 J/K, between about 19 J/K and 24 J/K, between about 20 J/K and 22 J/K, or about 21 J/K.

65. The humidifier system of claim 64, the base unit comprising a controller configured to detect one or more of a low water condition or a water-out condition based on a response of the disposable humidification chamber or the reusable humidification chamber, including any liquid contained therein, to a characteristic energization signal applied to the heater plate.

66. The humidifier system of claim 64 or 65, the reusable humidification chamber comprising the humidification chamber of any one of claims 1-13 or 27-60.
